(19) United States
(12) Patent Application Publication
(10) Pub. No.: US 2010/0282247 A1
(43) Pub. Date: Nov. 11, 2010

(54) TREATMENT OF PULMONARY DISORDERS WITH AEROSOLIZED MEDICAMENTS SUCH AS VANCOMYCIN

(75) Inventors: Nani P Kadrichu, San Carlos, CA (US); James B. Fink, San Mateo, CA (US)

Correspondence Address: NOVARTIS;CORPORATE INTELLECTUAL PROPERTY, ONE HEALTH PLAZA 101/2, EAST HANOVER, NJ, 07936-1080, US

(73) Assignee: Novartis AG, Basel (CH)

(21) Appl. No.: 12/679,464

(22) PCT Filed: Sep. 24, 2008
(86) PCT No.: PCT/US08/11137

§ 371 Date: Jul. 29, 2010

Related U.S. Application Data
(60) Provisional application No. 60/975,094, filed on Sep. 25, 2007.

(51) Int. Cl. A61M 11/00; A61K 9/12; A61K 38/14

(52) U.S. Cl. 128/200.14; 424/45; 514/20.9

(57) ABSTRACT

A method of administering an aerosolized anti-infective, such as a glycopeptide, to the respiratory system of a patient. A ratio of an amount of the glycopeptide, such as vancomycin, delivered to the pulmonary system of the patient in a 24 hour period to a minimum inhibitory amount for the target organ for the same period is about 2 or more. A system to introduce aerosolized medicament to a patient may include a humidifier coupled to an inspiratory limb of a ventilator circuit wye, where the humidifier supplies heated and humidified air to the patient, and an endotracheal tube having a proximal end coupled to a distal end of the ventilator circuit wye. The system may also include a nebulizer coupled to the endotracheal tube, where the nebulizer generates the aerosolized medicament.

### CROSS-REFERENCE TO RELATED APPLICATIONS

The present application claims priority from, and is a continuation in part of, U.S. Patent Application No. 60/975,094, filed Sep. 25, 2007.

The present application claims priority from, and is a continuation in part of, U.S. patent application Ser. No. 11/654,212, filed Jan. 16, 2007, which is a continuation-in-part of U.S. patent application Ser. No 11/090,328, filed Mar. 24, 2005, which is a continuation-in-part of Ser. No. 10/345,875, filed Jan. 15, 2003.

The present application additionally claims priority from, and is a continuation in part of, U.S. patent application Ser. No. 10/284,068, filed Oct. 30, 2002, which claims the benefit of 60/344,484 filed Nov. 1, 2001 and of 60/381,830 filed May 20, 2002, all of which are incorporated herein in their entirety.

The present application is also related to U.S. Patent Publication Nos. 2002-0134375; 2002-0134374; U.S. Pat. Nos. 6,948,491, 6,615,824, 6,968,840, and 7,100,600 and WO 2007/041156, the complete disclosures of which are incorporated herein by reference for all purposes.

### FIELD OF THE INVENTION

One or more embodiments of the present invention include systems and methods for the delivery of aerosolized medicaments such as anti-infectives. One or more embodiments of the present invention include systems and methods for the delivery of an aerosolized glycopeptide, such as vancomycin. One or more embodiments of the present invention include systems and methods for the pulmonary delivery of a glycopeptide, such as vancomycin. One or more embodiments of the invention relate to the coupling of aerosol generators with ventilator circuits and endotracheal tubes, permitting an aerosolized medicament, such as vancomycin, to be inhaled directly by a patient.

### BACKGROUND OF THE INVENTION

Aerosolized medicaments are used to treat patients suffering from a variety of respiratory ailments. Medicaments can be delivered directly to the lungs by having the patient inhale the aerosol through a tube and/or mouthpiece coupled to the aerosol generator. By inhaling the aerosolized medicament, the patient can quickly receive a dose of medicament that is concentrated at the treatment site (e.g., the bronchial passages and lungs of the patient). Generally, this is a more effective and efficient method of treating respiratory ailments than first administering a medicament through the patient's circulatory system (e.g., intravenous injection). However, problems still may exist with the delivery of aerosolized medicaments.

For example, respiratory disease is a major cause of morbidity and accounts for 90% of mortality in persons with Cystic Fibrosis (CF). CF patients suffer from thickened mucus caused by perturbed epithelial ion transport that impairs lung host defenses, resulting in increased susceptibility to early endobronchial infections with Staphylococcus aureus, Haemophilus influenzae, and Pseudomonas aeruginosa. By adolescence, a majority of persons with CF have P. aeruginosa present in their sputum. A link between acquisition of chronic endobronchial P. aeruginosa infection, lung inflammation, loss of lung function, and ultimate death is suggested by significantly decreased survival associated with chronic P. aeruginosa infections.

Tobramycin is approved for inhalation therapy for the treatment of endobronchial infections in CF patients. Administration of tobramycin for the suppression of P. aeruginosa in the endobronchial space of a patient is disclosed in U.S. Pat. No. 5,508,269, the disclosure of which is incorporated herein in its entirety by reference.

Limitations on the use of tobramycin in CF patients are created by issues related to the preparation and administration of nebulized tobramycin as well as the development of increased resistance, i.e. increase in minimal inhibitory concentration value (MIC) for P. aeruginosa during treatment.

Accordingly, the development of alternative inhaled antibiotic formulations which can be administered may provide CF patients a treatment alternative which does not require repopulation of susceptible pathogens and loss in pulmonary function.

Pneumonias, including those caused by Gram-negative bacteria and/or those caused by Gram-positive bacteria, are a persistent problem, especially with certain patient populations. Community acquired pneumonia (CAP) occurs throughout the world and is a leading cause of illness and death. Hospital acquired pneumonia (HAP), also sometimes called nosocomial pneumonia, is pneumonia acquired during or after hospitalization for another illness or procedure. A significant percentage of patients admitted to a hospital for other causes subsequently develop pneumonia. Hospitalized patients may have many risk factors for pneumonia, including mechanical ventilation, prolonged malnutrition, underlying heart and lung diseases. Hospital-acquired microorganisms may include resistant bacteria such as MRSA, Pseudomonas, Enterobacter, and Serratia. Ventilator acquired (or associated) pneumonia (VAP) may be considered a type of hospital-acquired pneumonia, as it occurs after intubation and mechanical ventilation. Other problematic pneumonias include SARS and idiopathic interstitial pneumonias, to name a few.

Pulmonary administration by nebulization of a liquid or powder is an ideal modality to treat infections, diseases and/or conditions of the lungs and pulmonary system, especially when respiratory function is diminished by disease or by injury. Lung diseases may be broadly grouped into obstructive diseases and restrictive diseases. In particular, the pulmonary system is susceptible to bacterial infections. Such infections may be treated with anti-infectives, including antibiotics.

Vancomycin is a tricyclic glycopeptide antibiotic that inhibits cell-wall biosynthesis in susceptible microorganisms. It also alters bacterial cell-membrane permeability and RNA synthesis. Vancomycin is active against several gram-positive pathogens, including both methicillin-sensitive Staphylococcus aureus (MSSA). Conventional means of administering vancomycin, however suffer from several drawbacks.

Patients who cannot breathe normally without the aid of a ventilator may only be able to receive aerosolized medicaments through a ventilator circuit. The aerosol generator should therefore be adapted to deliver an aerosol through the ventilator. Medicament delivery efficiencies for combination nebulizer-ventilator systems are, however, low, often dropping below 20%. The ventilator circuits typically force the aerosol to travel through a number of valves, conduits, and filters before reaching the patient's mouth or nose, and all the surfaces and obstacles provide an opportunity for aerosol particles to condense back into the liquid phase.

Conventional aerosolizing technology is not well suited for incorporation into ventilator circuits. Conventional jet and ultrasonic neublizers normally require 50 to 100 milliseconds to introduce the aerosolized medicament into the circuit. They also tend to produce aerosols with large mean droplet sizes and poor aerodynamic qualities that make the droplets more likely to form condensates on the walls and surfaces of the circuit.

Delivery efficiencies can also suffer when aerosols are being delivered as the patient exhales into the ventilator. Conventional nebulizers deliver constant flows of aerosol into the ventilator circuit, and the aerosol can linger, or even escape from the circuit when the patient is not inhaling. The lingering aerosol is more likely to condense in the system, and eventually be forced out of the circuit without imparting any benefit to the patient.

The failure of substantial amounts of an aerosolized medicament to reach a patient can be problematic for several reasons. First, the dosage of drug actually inhaled by the patient may be significantly inaccurate because the amount of medication the patient actually receives into the patient's respiratory system may vary with fluctuations of the patient's breathing pattern. Further, a significant amount of drug that is aerosolized may end up being wasted, and certain medications are quite costly, thus health-care costs are escalated.

Some of the unused medication may also escape into the surrounding atmosphere. This can end up medicating individuals in proximity to the patient, putting them at risk for adverse health effects. In a hospital environment, these individuals may be health-care providers, who could be exposed to such air pollution over a prolonged period of time, or other patients, who may be in a weakened condition or otherwise sensitive to exposure to unprescribed medications, or an overdose of a medication.

In addition to supplying medicament in a ventilator circuit, aerosolized medicaments are used to treat non-ventilated, and/or freely-breathing patients suffering from a variety of respiratory ailments. Medicaments can be delivered directly to the lungs by having the patient inhale the aerosol through a tube and/or mouthpiece coupled to the aerosol generator.

For these reasons, it's desirable to increase the aerosol delivery efficiency, and/or efficacy and or safety of nebulizer-ventilator systems as well as nebulizer systems to administer medicaments to freely-breathing patients.

Embodiments of the present invention address these and other problems with conventional systems and methods of treating patients with aerosolized medicaments.

It is to be understood that unless otherwise indicated the present invention is not limited to specific structural components, formulation components, drug delivery systems, manufacturing techniques, administration steps, or the like, as such may vary. In this regard, unless otherwise stated, a reference to a compound or component includes the compound or component by itself, as well as the compound in combination with other compounds or components, such as mixtures of compounds.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included.

As used herein and in the appended claims, the singular forms “a”, “an”, and “the” include plural referents unless the context clearly dictates otherwise. Thus, for example, reference to “a process” includes a plurality of such processes and reference to “the electrode” includes reference to one or more electrodes and equivalents thereof known to those skilled in the art, and so forth.

Also, the words “comprise,” “comprising,” “include,” “including,” and “includes” when used in this specification and in the following claims are intended to specify the presence of stated features, integers, components, or steps, but they do not preclude the presence or addition of one or more other features, integers, components, steps, acts, or groups.

“Anti-infective” is deemed to include antibiotics and antivirals, unless the context clearly indicates otherwise.

Reference herein to “one embodiment”, “one version” or “one aspect” shall include one or more such embodiments, versions or aspects, unless otherwise clear from the context.

As used herein, the terms “treating” and “treatment” refer to reduction in severity, duration, and/or frequency of symptoms, elimination of symptoms and/or underlying cause, reduction in likelihood of the occurrence of symptoms and/or underlying cause, and improvement or remediation of damage. Thus, “treating” a patient with an active agent as provided herein includes prevention or delay in onset or severity of a particular condition, disease or disorder in a susceptible individual as well as treatment of a clinically symptomatic individual.

As used herein, “effective amount” refers to an amount covering both therapeutically effective amounts and prophylactically effective amounts.

Fluid” means a liquid, or a gas, or a combination thereof, specifically including an aerosol.

“Medicament” comprises any drug, agent, vaccine, compound, biological material which beneficially treats, prevents, helps to prevent, mitigates or alleviates any disease or condition, unless the context clearly indicates otherwise.

As used herein, “therapeutically effective amount” refers to an amount that is effective to achieve the desired therapeutic result. A therapeutically effective amount of a given active agent will typically vary with respect to factors such as the type and severity of the disorder or disease being treated and the age, gender, and weight of the patient.

As used herein, the term “respiratory infections” includes, but is not limited to lower respiratory tract infections such as bronchiectasis (both the cystic fibrosis and non-cystic fibrosis indications), bronchitis (both acute bronchitis and acute exacerbation of chronic bronchitis), and pneumonia (including various types of complications that arise from viral and bacterial infections including hospital-acquired and community-acquired infections).

The entire contents and disclosure of each reference referred to herein, including US and PCT Patents and US and PCT Patent Application publications, is hereby incorporated herein by reference for all purposes.

### SUMMARY OF THE INVENTION

Accordingly, one or more embodiments of the present invention comprise anti-infective compositions, methods of making and using such compositions, and systems for pulmonary delivery of such compositions.

One or more embodiments of the present invention comprise glycopeptide and/or lipoglycopeptide anti-infective compositions, methods of making and using such compositions, and systems for pulmonary delivery of such compositions.

One or more embodiments of the present invention comprise compositions comprising vancomycin, methods of making and using such compositions, and systems for pulmonary delivery of such compositions.

The present invention contemplates drugs and drug combinations that will address a wide variety of conditions caused by a wide variety of organisms. In one or more embodiments, the present invention contemplates drugs or drug combinations effective in the treatment of infections caused by one or more of P. aeruginosa, S. aureus, H. influenza, and S. pneumoniae, Acinetobacter species, and/or antibiotic-resistant strains of bacteria such as methicillin-resistant S. aureus, among others.

One or more embodiments of the invention provide treatments for a variety of ailments using a variety of aerosolizable medicaments. The ailments may comprise pulmonary ailments such as ventilator-acquired pneumonia (VAP), hospital-acquired pneumonia (HAP), community- acquired pneumonia (CAP), mycobacterial infection, bronchitis, Staph infections including MRSA, fungal infections, viral infections, protozal infections, and acute exacerbation of Chronic Obstructive Pulmonary Disease, among others.

One or more embodiments of the invention relate to compositions and methods for treating bacterial infections. One or more embodiments comprise compositions and methods for the treatment of Cystic Fibrosis (CF). One or more embodiments comprise compositions and methods for the treatment of Pneumonias, such as VAP, HAP or CAP.

One or more embodiments of the invention include a method of administering an aerosolized glycopeptide, such as vancomycin to a patient. The methods may include the steps of converting the glycopeptide into a liquid aerosol, and delivering the aerosolized glycopeptide to the respiratory system of the patient.

One or more embodiments of the invention include methods of administering an aerosolized glycopeptide intermittently to a ventilator circuit. The glycopeptide may be administered singly, or in combination with other anti-infectives (including other glycopeptides and/or aminoglycosides).

One or more embodiments of the invention include methods of administering an aerosolized glycopeptide to a free-breathing patient using a portable aerosolizer. The glycopeptide may be administered singly, or in combination with other anti-infectives (including other glycopeptides and/or aminoglycosides).

One or more embodiments of the invention include methods of administering an aerosolized glycopeptide to a free-breathing patient using a portable aerosolizer and aerosolization chamber. The glycopeptide may be administered singly, or in combination with other anti-infectives (including other glycopeptides and/or aminoglycosides).

One or more embodiments of the invention include methods of administering an aerosolized liquid vancomycin to a patient wherein a ratio of an amount of the vancomycin delivered to the patient in a 24 hour period to a minimum inhibitory amount for the same period of 2 or more provides a therapeutic effect.

Embodiments of the invention may also comprise methods of administering vancomycin to a patient. The methods may include the steps of converting the vacomycin into a liquid aerosol, and delivering the aerosolized vancomycin continuously to a ventilator circuit coupled to the respiratory system of the patient.

The ratio of an amount of the vancomycin delivered to the patient's target organ (i.e. lungs and/or trachea and/or or pulmonary system) in a 24 hour period to a minimum inhibitory amount for the same period may be about 2 or more, such as 3 or 4 or 5 or 8 or 10 or 15 or 20 or 25 or 30 or 40 or 50 or more.

The ratio of an amount of the vancomycin delivered to the bronchial and/or pulmonary system of a patient in a 12 hour period to a minimum inhibitory amount for the same period may be about 2 or more, such as 3 or 4 or 5 or 8 or 10 or 15 or 20 or 25 or 30 or 40 or 50 or more.

Embodiments of the present invention include one or more methods for adjunctive therapy, wherein an amount of glycopeptide, such as vancomycin, administered to a patient by means other than inhalation is reduced.

Embodiments of the present invention include one or more methods for adjunctive therapy, wherein a therapeutically-effective amount of glycopeptide, such as vancomycin, administered to a patient by means other than inhalation is reduced by at least about 40%, such as 50% or 60% or 70% or 80% or more.

Embodiments of the present invention include one or more methods for adjunctive therapy, wherein the number of days a patient is required to receive a therapeutically-effective glycopeptide, such as vancomycin, administered to a patient by means other than inhalation, is reduced.

Embodiments of the present invention include one or more methods for administration of aerosolized antibiotics to a patient wherein an glycopeptide, such as vancomycin, concentration in epithelial lining fluid, or tracheal aspirates, or both, exceeds a minimum inhibitory concentration for microorganisms usually responsible for Gram-positive pneumonia.

Embodiments of the present invention include one or more methods for administration of aerosolized antibiotics to a patient wherein an glycopeptide, such as vancomycin, concentration in epithelial lining fluid, or tracheal aspirates, or both, exceeds at least about four times a minimum inhibitory concentration for microorganisms usually responsible for Gram-positive pneumonia.

Embodiments of the present invention include one or more methods for administration of aerosolized glycopeptide, such as vancomycin, to a patient wherein an glycopeptide, such as vancomycin, concentration in the lung and/or pulmonary system is present in a therapeutic-effective amount and a maximum glycopeptide, such as vancomycin, concentration in the blood serum is at least about 3 times lower, such as 5 or 10 or 20 or 50 or 100 or more times lower.

Embodiments of the present invention include one or more methods for administration of aerosolized vancomycin to a patient wherein a vancomycin concentration in the lung and/or pulmonary system is present in a therapeutic-effective amount and a maximum vancomycin concentration in the blood serum is less than about 40 μg/mL, or a trough level is below about 15 μg/mL, or both.

Embodiments of the present invention include one or more methods for administration of aerosolized vancomycin to a patient wherein a vancomycin concentration in the lung and/or pulmonary system is present in a therapeutic-effective amount, such as about 128 μg/mL and a maximum vancomycin concentration in the blood serum is less than about 40 μg/mL, or a trough level is below about 15 μg/mL, or both

Embodiments of the present invention include one or more methods method of treating a patient with a pulmonary disease, wherein the method comprises administering an aerosolized first medicament comprising vancomycin to the patient and administering, systemically a second medicament comprising an antibiotic to the patient that also treats the pulmonary disease, wherein a resulting vancomycin concentration in the lung and/or pulmonary system is therapeuticlly-effective, and an amount of the systemically administered second antibiotic is reduced.

Embodiments of the present invention include one or more methods for administration of aerosolized glycopeptides to a patient wherein a glycopeptide concentration in the lung and/or pulmonary system is present in a therapeutic-effective amount, and a need for systemically administered antibiotics is reduced.

Embodiments of the present invention include one or more methods for administration of aerosolized antibiotics to a patient wherein a glycopeptide is dispersed into the deep lung and/or peripheral regions to provide a therapeuticlly-effective amount thereto.

One or more embodiments of the invention comprise systems and method for delivering relatively high concentrations of medicament without undue or significant precipitation of the medicament in the delivery system.

One or more embodiments of the invention comprise systems and methods for delivering aerosolized doses of relatively high concentrations of medicament, such as vancomycin, wherein less than about 50% or 40% or 30% or 20% of the starting dose of the medicament precipitates in the delivery system.

Embodiments of the invention may further include methods of treating a patient with a pulmonary disease. The methods may include administering to the patient a nebulized aerosol comprising a glycopeptide such as vancomycin, where the nebulized aerosol is delivered to the patient though a ventilator circuit. At least about 30% or 40% or 50% of the aerosol may be transmitted through the ventilator circuit to the patient.

Embodiments of the invention may still also include methods of introducing aerosolized glycopeptide, such as vancomycin to a patient. The methods may include coupling a nebulizer between a ventilator circuit wye and an endotracheal tube, and supplying a liquid or powdered medicament comprising vancomycin to the nebulizer. The nebulizer generates the aerosolized vancomycin from the supplied medicament. The methods may also include mixing the aerosolized glycopeptide, such as vancomycin with humidified and heated air, wherethe air carries at least a portion of the aerosolized vancomycin to a lung of the patient.

One or more embodiments of the invention comprise an aerosolized drug delivery system comprising a programmable controller, which controller may be programmed to optimize delivery of a given drug.

One or more embodiments of the invention comprise an aerosolized drug delivery system comprising a programmable controller, and a drug container comprising a signaling or keying means to uniquely identify the drug to the controller, permitting the controller to optimize delivery of the drug. Such means may include a wireless (RF) subsystem, optical or mechanical signaling means, or combinations thereof. A drug container may be equipped with an RFID tag, for example, configured to provide drug information to the controller to optimize aerosolization for efficiency, efficacy, safety or combinations.

Embodiments of the invention may also further include systems to introduce aerosolized medicament to a patient. The systems may include a humidifier coupled to an inspiratory limb of a ventilator circuit wye, where the humidifier supplies heated and humidified air to the patient. They may also include an endotracheal tube having a proximal end coupled to a distal end of the ventilator circuit wye, and a nebulizer coupled to the endotracheal tube. The nebulizer generates the aerosolized medicament from a medicament source supplied to the nebulizer. The medicament source may be an aqueous liquid solution comprising vancomycin or a powdered solid comprising vancomycin, among other medicaments.

One or more embodiments of the invention include methods of administering an aerosolized glycopeptide to a patient for treatment or prophalaxis of a disease or condition. The glycopeptide may be administered singly, or adjunctively with other anti-infectives (including other glycopeptides and/or aminoglycosides). The adjunctive administration may be serial or concurrent, and may further include at least one other form of administration, such as oral, intramuscular, intraveneous etc.

Further embodiments comprise any two or more of any of the features, aspects, versions or embodiments, supra or infra.

Additional embodiments and features are set forth in part in the description that follows, and in part will become apparent to those skilled in the art upon examination of the specification or may be learned by the practice of the invention. The features and advantages of the invention may be realized and attained by means of the instrumentalities, combinations, and methods described in the specification.

### BRIEF DESCRIPTION OF THE DRAWINGS

FIG. 1 illustrates components of a pulmonary drug delivery system according to embodiments of the invention;

FIG. 2 is a perspective view of an embodiment of an aerosolization and aerosol transfer device of the present invention;

FIG. 3 is a side elevational view of an embodiment of the aerosolization and aerosol transfer device of FIG. 2;

FIG. 4 is a perspective view of another embodiment of an aerosolization and aerosol transfer device of the present invention;

FIG. 5 shows a nebulizer coupled to T-piece adaptor for a ventilator circuit according to embodiments of the invention;

FIG. 6 shows an exploded view of a nebulizer according to embodiments of the invention;

FIG. 7 is a schematic cross-sectional representation of an aerosol generator in accordance with embodiments of the present invention;

FIG. 8 is a schematic cutaway cross-section detail of the aerosol generator represented in FIG. 7;

FIGS. 9A-B are exploded perspective views of embodiments of a vibration system of the present invention.

FIG. 10 is a partial cross-sectional view of the assembled vibration system of FIGS. 9A-B.

FIG. 11 shows an embodiment of an aerosolization chamber according to the present invention;

FIGS. 12A-C are graphs of various modes of aerosolization over the course of breathing cycles;

FIG. 13 is a flowchart illustrating a simplified method according to embodiments of the present invention;

FIG. 14 is a schematic representation of algorithms of operating sequences in accordance with embodiments of the present invention;

FIG. 15 is a schematic representation of algorithms of operating sequences in accordance with embodiments of the present invention;

FIG. 16 is a further schematic representation of algorithms of operating sequences shown in FIG. 15, and in accordance with the present invention;

FIG. 17 is a schematic representation of an algorithm by which an operating sequence may be chosen based on a combination of a plurality of independent sets of information;

FIG. 18A shows a conventional experimental setup for testing the delivery efficiency of an aerosolized medicament (e.g., vancomycin) to a patient;

FIG. 18B shows a modification of the conventional experimental setup in FIG. 18A by placing a test lung and filter above the endotracheal tube (EU) and adding two traps;

FIG. 18C shows a further modification of the experimental setup in FIG. 18B by adding a humidifier between the test lung and filter;

FIG. 18D shows a further modification of the experimental setup in FIG. 18C by replacing the mechanical test lung with a simple bag lung;

FIG. 19 is a graph showing delivered dose v flow rate for three different concentrations of vancomycin hydrochloride;

FIG. 20 is a graph that shows delivery efficiency (i.e., percent of medicament delivered to the patent) under Setups 2, 3 and 4 (FIGS. 18B-D, described infra);

FIG. 21 is a graph that shows the deposition of aerosol (mean±SD) in each of the 8 Compartments in Setup 4 (FIG. 18D) with peak inspiratory flows of 80 and 40 Lpm, and heat and humidity On and Off;

FIG. 22 shows a simplified schematic of a system to introduce an aerosolized medicament, such as vancomycin, to a patient according to embodiments of the invention; and

FIG. 23 shows a graph drug concentration versus time to illustrate Pharmacokinetic/Pharmacodynamic efficacy prediction factors.

### DETAILED DESCRIPTION OF THE INVENTION

As noted above, conventional nebulizer-ventilator systems have low medicament delivery efficiency (e.g., less than 20%). Embodiments of the invention include methods and systems for increasing delivery efficiencies to, for example, at least 25% or at least 30% or at least 40%, at least 50%, at least 60%, at least 70%, at least 80%, or more. The increased efficiency for delivering the aerosolized medicament may be attributable, in part, to one or more features that may be implemented in embodiments of the invention. These features include synchronizing the generation of aerosol with an inspiratory phase of the ventilator cycle (e.g., phasic delivery). The features may also include supplying air (e.g., an “air chaser”) following aerosol generation, which can clear the endotracheal tube and reduce the amount of medicament exhaled by the patient. Features may further include connecting the aerosol generating unit directly to the hub of the endotrcheal tube that is connected to the patient. Still other features include generating aerosolized medicament with smaller particle sizes (e.g., about 1 to 7 microns (μm) average diameter). Additional features may also include storing the medicament in a conical shaped reservoir to minimize the residual medicament volume.

Embodiments of the present invention include methods for improved drug delivery by aerosol generator placement at the endotracheal tube and further include humidification of the airway. In some embodiments, an active humidifier is placed between the lung and inspiratory filter. This reduces the variability in inhaled dose between wet and dry conditions and improved quantification of inhaled versus instilled dose.

A method to quantify aerosol delivered beyond the distal tip of the ETT, is thus provided by systems, and circuits to differentiate drug delivered as aerosol from that of liquid that may have “dripped” down the airway into the filter. This liquid can be a combination of drug from aerosol impacted within the airway and water vapor condensation forming in the airway upon leaving the heated, humidified ventilator circuit.

Embodiments may include placement of a nebulizer between the ventilator and the ETT proximal to airway that may result in more of a medicament (e.g., a liquid containing drug) delivered through the ETT than a conventional nebulizer placement in the inspiratory limb of the ventilator circuit. An optimization of the structure, components and orientation resulted in enhanced medicament delivery to the lung, in particular with a nebulizer placed between the ventilator circuit in close proximity to the ETT (e.g., coupled to the distal end of the ETT), under heated/humidified conditions. The delivery efficiency of aerosolized medicaments with these component configurations may result in delivery of, for example, at least 40%, or 50% or 60% or 70% or 80% or more, of the aerosolized medicament to the patient.

Embodiments of the invention comprise nebulizer ventilator systems which allow high delivered concentrations of medicament without significant precipitation of medicament, as is often found in systems of the prior art. This is particularly advantageous when the medicament comprises a glycopeptide, such as vancomycin, or lipoglycopeptide, such as dalbavancin, as the efficaciousness of the medicament is highly time-concentration dependent.

Embodiments of the systems are configurable to administer aerosolized medicament to a patient both on-ventilator and off-ventilator. On-ventilator treatment methods include administering the nebulized aerosol through a ventilator circuit to the patient. Aerosol doses, containing, for example, about 1 to about 500 mg of a medicament, may be delivered through the ventilator circuit in a phasic or non-phasic manner. Off-ventilator treatment methods may include taking the patient off the ventilator before administering the nebulized aerosol. Once the treatment session is completed the patient may be put back on the ventilator, or may breathe on his or her own without assistance.

Embodiments of the invention provide treatments for a variety of ailments using a variety of aerosolizable medicaments. The ailments may include pulmonary ailments such as ventilator-associated pneumonia, hospital-acquired pneumonia, cystic fibrosis, mycobacterial infection, bronchitis, staph infection, fungal infections, viral infections, protozal infections, and acute exacerbation of Chronic Obstructive Pulmonary Disease, among others. The aerosolizable medicaments used to treat the ailments may include antibiotics, anti-oxidants, bronchodialators, corticosteroids, leukotrienes, protease inhibitors, and surfactants, among other medicaments.

### Exemplary Pulmonary Drug Delivery Systems

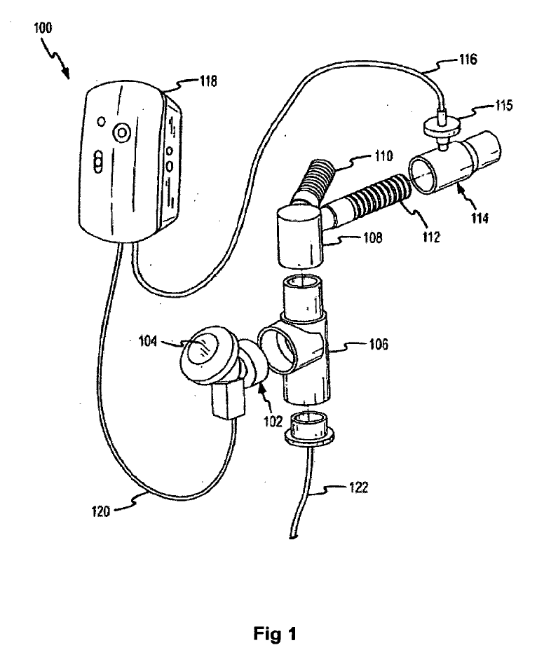

FIG. 1 shows an embodiment of a pulmonary drug delivery system (“PDDS”) 100 according to the invention. The PDDS 100 may include a nebulizer 102 (also called an aerosolizer), which aerosolizes a liquid medicament stored in reservoir 104. The aerosol exiting nebulizer 102 may first enter a T-adaptor 106 that couples the nebulizer 102 to a ventilator circuit. One embodiment of such a T-adapter is described in co-owned, pending U.S. application Ser. No. 11/990,587. If present, the T-adaptor 106 is also coupled to the circuit wye 108 that has branching ventilator limbs 110 and 112. In this example limb 110 is expiratory and 112 is inspiratory.

Coupled to the ventilator limb 112 may be an air pressure feedback unit 114, which may act to equalize the pressure in the limb with the air pressure feedback tubing 116 connected to a control module 118. In the embodiment shown, feedback unit 114 has a female connection end (e.g., an ISO 22 mm female fitting) operable to receive ventilator limb 112, and a male connection end (e.g., an ISO 22 mm male fitting) facing opposite, and operable to be inserted into the ventilator. The feedback unit may also be operable to receive a filter 115 that can trap particulates and bacteria attempting to travel between the ventilator circuit and tubing 116.

The control module 118 may monitor the pressure in the ventilator limb via tubing 116, and use the information to control the nebulizer 102 through system control cable 120. In other embodiments (not shown) the control module 118 may control aerosol generation by transmitting signals to a complementary control module on the nebulizer 102. Such signals may be wireless (RF), optical or other.

During the inhalation phase of the patient's breathing cycle, aerosolized medicament entering T-adaptor 106 may be mixed with the respiratory gases from the inspiratory ventilator limb 112 flowing to the patient's nose and/or lungs. In the embodiment shown, the aerosol and respiratory gases flow through endotracheal tube 122 (which may also be configured as a nasal cannula or mask) and into the pulmonary system of the patient.

Other configurations, aspects, versions or embodiments of the circuit 108 shown in FIG. 1 are also contemplated embodiments of the invention. These configurations, aspects, versions and embodiments are fully disclosed and described in co-owned US Patent Applications 2005/0217666, 2007/0083677, and 2005/0139211.

In one example the junction device provides for the gas flow (containing aerosolized medicament) to follow a straight unobstructed path through the respiratory circuit without any portion being diverted. In other words, there is virtually no change in the angle of the path of gas flow. As a result, the full amount of aerosol particles of medicament contained in gas flow is efficiently delivered through the respiratory circuit to the patient.

PDDS systems of the present invention may include equipment for phasic delivery of aerosolized medicaments. This equipment may include breathing characteristics sensors, which can monitor the breathing characteristics of a patient using the PDDS. The sensors can send breathing characteristic information to the PDDS controller to allow the controller to select an appropriate delivery cycle of the aerosolized liquid to the patient. Typically, breathing characteristic sensors can be used to measure one or more characteristics of a breathing pattern of the patient, such as a peak flow, breathing rate, exhalation parameters, timing, regularity of breathing, flow volume, pressure changes, and the like. Such measured breathing characteristics data may be delivered to controller by analog or digital signals, and run through a software algorithm to determine an appropriate sequence of delivery of aerosolized medicament to the patient relative to one or more of the measured characteristics.

One exemplary breathing characteristic that may be sensed by a sensor is the cycle of a ventilator providing air to a patient; for example, the start of an inhalation cycle generated by the ventilator. The sensor may additionally or alternatively sense other parameters, for example, it may be an acoustic sensor that is activated through passing the respiratory flow of the patient through an acoustic chamber so as to produce an acoustic tone, which is proportional to the inspiratory flow rate. The frequency of the acoustic tone indicates the inspiratory flow rate at any instant of the breathing cycle. The acoustic signal can be detected by the controller such that integration of the flow rate with time produces the tidal volume. Both the flow rate and the tidal volume can then be used by the controller to determine when the aerosol generator generates the droplets and at what mass flow rate such that maximum deposition of droplets is obtained. Further, the acoustic tone may be recorded to produce a record of the breathing pattern of the patient which may be stored in the microprocessor. This information can be later used to synchronize the ejection of droplets for the same patient. Such information may also be later employed for other diagnostic purposes. A more complete description of such sensors may be found in commonly owned, U.S. Pat. No. 5,758,637, to Ivri et al., incorporated herein by reference.

In some embodiments, one or more sensors can be used to monitor the breathing characteristics of the patient throughout the delivery regime so as to ensure that the aerosol is efficiently delivered throughout the aerosolization procedure. In such embodiments, the controller can adjust the aerosol delivery based on any measured or and/or calculated change in the breathing pattern of the patient during the aerosolization. With this monitoring and adjustment predetermined times for the beginning and ending of aerosolization can be reset based on the actual breathing of the patent. In other embodiments, however, the breathing sensor can be used to determine the breathing cycle of a tidal breath and to choose the appropriate pre-programmed delivery cycle that is stored in the memory of the controller. In other embodiments, the controller may be configured to provide aerosol based on the time. For example, the controller may be configured to start aerosol production at the beginning of an inhalation phase of a breathing cycle and stop at a point at which a predetermined percentage of the inhalation has taken place.

Additionally or alternatively, the controller may be configured to start aerosolization at a first point at which a first predetermined percentage of inhalation has taken place, and stop aerosolization at a second point at which a second predetermined percentage of that inhalation has taken place. Additionally or alternatively, aerosol may begin during an inhalation phase and end during the subsequent exhalation phase. Additionally or alternatively, the controller may be configured to begin aerosol production at a certain point during exhalation and stop during that exhalation or during the subsequent inhalation.

Thus, in one or more embodiments the PDDS may include a nebulizer having an aerosol generator and a controller configured to have the controller begin aerosolization during exhalation and stop during the same exhalation or in a subsequent inhalation. For example, aerosolization may begin after about the 30% point in an expiration cycle and may continue until about the 75% point in the expiration cycle. These values may range (in any combination) as beginning after about 35 or 40 or 45 or 50 or 55 or 60% of the expiration cycle and continuing until about 50 or 55 or 60 or 65 or 70 or 75 or 80 or 85 or 90 or 95% of the expiration cycle.

In still other embodiments, the controller may be configured to begin aerosol production at a start point in the breathing cycle, and continue to generate aerosol for a set period of time regardless of how a patient's breathing cycle varies. At the end of the time period, aerosol generation stops until the next start point is in the breathing cycle.

In further embodiments, the controller may be configured to start and stop aerosol production for preprogrammed periods of time that are independent of the patient's breathing cycle. Such protocols may be useful, for example, in high-frequency oscillation ventilation, and in jet ventilation.

In one or more embodiments, the controller may be operable to allow for a choice of modes of operation, for example, a mode in which aerosolization begins once a certain breath characteristic is detected, such as a sufficient level of inhalation, and ends when there is no longer a sufficient level; another mode in which aerosolization begins once a certain breath characteristic is detected, such as a sufficient level of inhalation, and ends at a predetermined time within the inhalation cycle, such as for example, before the level of inhalation falls below that required for operation of an aerosolization element, and/or, alternatively, at any other point within the inhalation cycle, such as after the inhalation phase of the cycle before exhalation has begun, or after exhalation has begun.

In one or more embodiments, the level of inhalation may be sensed by a pressure sensor. Such a transducer may monitor a drop in air pressure or a rise in air pressure within a chamber that is in fluid communication with the ventilator circuit. In this manner, a pressure drop may be sensed by a patient inhaling through the circuit, for example, in an instance in which the ventilator provides assisted ventilation initiated by a patient's commencement of an inhalation. Similarly, a pressure rise may be sensed in an instance in which the ventilator pushes inhalation air to the patient without the patient initiating a breath. Another mode in which the controller may be operable is a mode in which the on/off operation of the aerosol generator is triggered by time, which may be ascertained from an internal clock device, such as a clock built into a microprocessor, or from an external source.

Yet another mode in which the controller may be operable is in which the on/off operation of the aerosol is triggered by the controller receiving an external signal, such as a signal from a ventilator, which can correspond to the point in the ventilator's cycle of that is the start of an inhalation phase in which the ventilator begins to push inspiratory air into the ventilator circuit. The controller may be operable based upon a single sensory mode, or combinations of sensory modes. The controller may be operable between such modes, including a mode in which the aerosolization begins at a predetermined time in the breathing cycle and ends at a predetermined time in the breathing cycle. The first and second predetermined times in the third mode may be during inhalation. Alternatively, or additionally, the first and second predetermined times may be during exhalation, or at the first predetermined time may be during exhalation and the second predetermined time may be during subsequent inhalation. These times may correspond to certain percentages of the inhalation phase taking place, or any other points of reference within a breathing cycle.

Alternatively, or additionally, the first predetermined time and the second predetermined time may be designated as any point within a single breathing cycle, or alternatively, the first predetermined point may be at any point within one breathing cycle and the second predetermined point may be at any point in a subsequent breathing cycle. The controller may make the determination of when to begin, and operate to begin aerosolization, and may make the determination of when to stop aerosolization to stop, and cause aerosolization to stop. The controller may make such determinations and take such actions based on accessing stored algorithms. The controller may receive a signal from the ventilator that establishes a reference point, nonetheless, the controller, by making the determinations an taking the actions based on stored algorithms, and/or information obtained as to the identity of a drug to be administered, may cause aerosol production to begin and/or end independent of the instantaneous position of the ventilator with respect to the ventilator cycle.

Embodiments also include a controller operable to allow for a single mode of operation, where the single mode of operation may be any mode, including but not limited to the modes described above. For example, a mode in which aerosolization begins once a certain breath characteristic is detected, such as a sufficient level of inhalation, and ends when there is no longer a sufficient level. Similarly, the controller may operable in a mode in which aerosolization begins once a certain breath characteristic is detected, such as a sufficient level of inhalation, and ends at a predetermined time within the inhalation before there is no longer a sufficient level or an aerosolization element.

Alternatively or additionally, the mode may be a mode in which the aerosolization is commenced based on a signal from the ventilator indicating the attainment of a certain point within the ventilation output cycle or the inhalation cycle of the patient. The ventilation output cycle of the ventilator may coincide with the inhalation cycle of the patient, such that the ventilation output phase of the ventilator output cycle and the inhalation phase of the inspiratory cycle of the patient occur substantially simultaneously. Such may be the case where a patient is completely passive and the only inhalation that occurs is by generation of air from the ventilator during the output phase of the ventilator cycle. Such point may be during the output phase of the output cycle of the ventilator or during the inhalation phase of the inhalation cycle of the patient. The predetermined point can be chosen to coincide with a certain level of output from the ventilator or at a certain point in time during the ventilator output cycle. Such a predetermined point may be a specific point within the output phase of the ventilator cycle, or, a specific point within the non-output phase of the ventilator cycle, based, for example, on the timing of the previous or succeeding output phase of the ventilator. In other aspects, the present invention provides for a ventilator along with the aerosol generator and controller. In an aspect of the invention, a predetermined time may be based on the timing of a ventilator supplying air to a user. In this manner, the controller may be set to work off of the timing of the ventilator in one mode, while working off the patient's inspiratory effort in another mode, or mode that allows for a combination of the patient's inspiratory effort and the timing of the ventilator, for example, where the ventilator is set to assist the patient by supplying air upon the patient's effort or where the patient has not made a sufficient effort within a predetermined period of time.

Embodiments of the invention further include a controller operable to allow for two or more modes of operation, where any single mode of operation may be combined with any other mode, including but not limited to the modes described above. Similarly, embodiments of the invention further include a controller operable to allow for multiple modes of operation.

### Exemplary Off-Ventilator Configurations

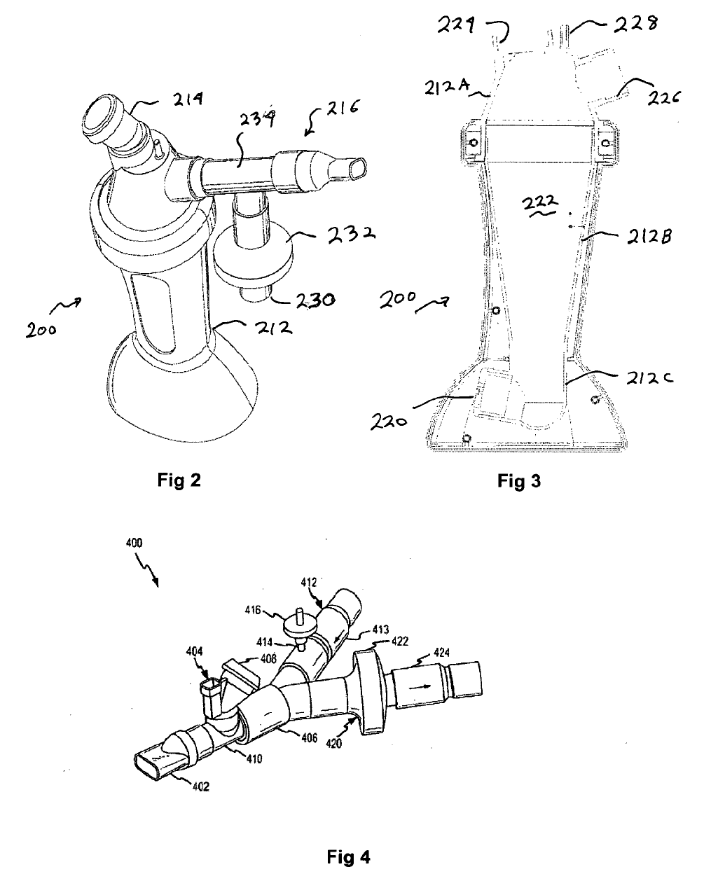

Referring now to FIGS. 2-3 one or more embodiments of an off-ventilator, or handheld, configuration of a PDDS is shown. The apparatus comprises a hand held aerosolization transfer/accumulation system identified by the general reference numeral 200. The system 200 comprises an aerosolization chamber or body 212 (also sometimes referred to herein as an accumulator), a nebulizer 214 and a patient interface 216. The nebulizer 214 (also sometimes referred to as an aerosol generator) comprises the source of aerosol which is thereby discharged into the body 212. The patient/aerosol generator interface 216 comprises the output for the generated aerosol, and is the means by which the aerosol is transported from the body 212 to the patient. The patient interface 216 may comprise a variety of structures, such as a mask, mouthpiece, hood, helmet, chamber, nosepiece, mechanical ventilator circuit, intubation catheter and tracheal catheter.

As illustrated also in FIG. 3, the body 212 may be conveniently subdivided into three components: an upper body 212A, an intermediate body 212B and a lower body 212C. In one or more embodiments of the upper body 212A is fluidically coupled to the nebulizer 214, and to the patient interface 216. In one or more embodiments, the lower body 212C comprises an ambient air inlet 220. In one or more embodiments, the intermediate body 212B fluidically connects the upper body 212A and the lower body 212C. The intermediate body 212B is shaped and configured to optimize mixing of ambient air from the inlet 220 and the aerosol generated by the nebulizer 214, resulting in the formation of an aerosol plume having optimum characteristics for delivery of the aerosol to the patient's pulmonary system, such as the central or deep lung regions. The shape and dimensions of the body 212B are further designed to minimize aerosol deposition in the system 200, thus improving delivery efficiency, as determined, for example by inhaled mass, and/or by target lung dose, and/or by pharmacokinetics. In one or more embodiments, the body 212 has a length, or a width, or both that is greater than the corresponding length, or width, or both of the aerosol plume.

In one or more embodiments, the aerosol plume is low velocity. In one or more embodiments, the aerosol plume has an initial velocity (immediately downstream of the aerosol generator 214) of between about 0.5 and 8 meters per second (m/s). Typically, such a plume decelerates rapidly after generation.

Aerosol generated by the nebulizer 214 is delivered into an aerosolization chamber 222 (FIG. 3) defined by the bodies 212A, 212B and 212C. The body 212A is provided a nebulizer inlet port 224, and an aerosolized medicament outlet port 226 and may include a fluid control port 228 to which a fluid coupling may be connected.

Air is admitted via aperture 220 into the chamber 222 and thereby entrains the aerosolised medicament generated by the nebulizer 214. The air/medicament aerosol mixes in the chamber 222, and which is then delivered to the patient through the aerosolized medicament outlet port 226 via the patient interface device 216. An exhalation exhaust port 230 and a filter 232 may located on a tube 234 at a point intermediate to the body 212A and patient interface device 216. Preferably the filter 232 and exhaust port 230 are oriented to have an upward component.

In one or more embodiments, the aerosol generator is controlled by an electronic controller, such as that described in greater detail in U.S. Pat. Nos. 6,540,154, 6,546,927 and 6,968,840 and US Patent Application Publication 2005/0217666, published Oct. 6, 2005.

In one or more embodiments, it is sufficient that the controller supply power to the piezoelectric generator and to switch generation of the aerosol on and off between patients. In other embodiments, the controller may supply power and switch the aerosol generator 214 on and off according to a predefined protocol or according to measured or calculated breathing characteristics, or both. For example a pressure sensor (not shown) may be fitted to port 228 in the nebulizer body 212, and used to measure breathing characteristics.

In one or more embodiments of the invention, the nebulizer 214 operates continuously and during the patient's expiration phase, the aerosol continues to be produced and is stored in chamber 222 for the next inhalation. This mode of operation affords simplicity and efficiency in administration, as patient breathing characteristic do not need to be measured, nor does the controller require extensive circuitry responsive to such measurements. In one or more embodiments, the nebulizer 214 operates continuously, except that a controller (for example, the controller 118 of FIG. 1) includes a shut-off means to shut off the nebulizer 214 if and when the patient interrupts breathing into the interface device 216. The shut off means could, in some embodiments, comprise a simple pressure or flow sensor, and appropriate control circuitry. In one or more embodiments, the nebulizer can be operated intermittently, and/or phasically, and/or be breath actuated, such that aerosol is dispensed and/or inhaled in different patterns as related to a given patient's inspiratory and/or expiratory cycles.

Additional description and disclosure of an embodiment of a handheld, or off-ventilator aerosolization system are found in commonly-owned U.S. Patent Application 61/123,133, to Fink et al., filed 4 Apr. 2008.

Other exemplary off-ventilator configurations are shown in commonly-owned US Patent Application Publication 2005/0217666 to Fink et al.

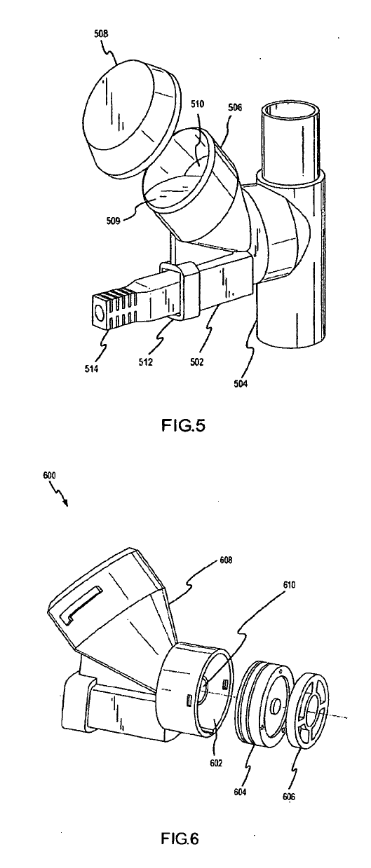

Other versions of an off-ventilator nebulization system, such as a PDDS are illustrated in FIG. 4, and designated by the general reference character 400. The system 400 includes an endpiece 402 that is coupled to a nebulizer 404 and wye 406. The nebulizer 404 may include reservoir 408, which supplies the liquid medicament that is aerosolized into connector 410. The connector 410 can provide a conduit for the aerosolized medicament and gases to travel from the wye 406 to endpiece 402, and then into the patient's mouth and/or nose. The first wye limb 412 may be connected to a pump or source of pressurized respiratory gases (not shown), which flow through the wye limb 412 to the endpiece 402. A one-way valve 413 may also be placed in the limb 412 to prevent respired gases from flowing back into the pump or gas source. The limb 412 may also include a pressure feedback port 414 that may be connected to a gas pressure feedback unit (not shown). In the embodiment shown, a feedback filter 416 may be coupled between the port 414 and feedback unit.

The pressure in the system may be monitored throughout the breathing cycle with a pressure sensor coupled to pressure port 416. The pressure sensor (not shown) may generate an analog or digital electronic signal containing information about the pressure level in the apparatus. This signal may be used to control the amount of aerosolized medicament and/or gases entering the apparatus over the course of the patient's breathing cycle. For example, when the pressure in the apparatus decreases as the patient inhales, the pressure signal may cause the nebulizer 404 to add aerosolized medicament to the apparatus, and/or cause the gas source or pump to add gas through inlet 412. Then, when the pressure in the apparatus increases as the patient exhales, the pressure signal may cause the nebulizer 404 to stop adding aerosolized medicament to the apparatus, and/or cause the gas source or pump to stop adding gas through inlet 412. Controlling the aerosol and/or gas flow based on the patient's breathing cycle, i.e., phasic delivery of the gases and aerosols, will be described in additional detail below.

The off-ventilator PDDS 400 may additionally or alternatively include a filter 422 and one-way valve 424, through which gases may pass during an exhalation cycle. The filter 422 may filter out aerosolized medicament and infectious agents exhaled by the patient to prevent these materials from escaping into the surrounding atmosphere. The one-way valve 424 can prevent ambient air from flowing back into the PDDS 400.

Other configurations of an off-ventilator PDDS 400, comprise replacing the endpiece 402 with a mouthpiece (not shown) or a mask (not shown), operable to sealingly engage the lips of a patient. The mouthpiece or mask may be made from an elastomeric material (e.g., rubber, silocone, etc.) that can resiliently couple the mouthpiece to the system 400. A gas inlet port (not shown) may be provided to permit an external source of gas, such as oxygen, to be inhaled with the medicament.

The on and off-ventilator configurations of the PDDS allow continuity of treatment as the patient switches between on-vent and off-vent treatment configurations. In both configurations, a patient is able to receive the same aerosolized medicament at the same dosage level, providing a continuity of treatment as the patient transitions from on-ventilator care to off-ventilator care. This can be particularly useful for extended treatment regimens, when the patient receives the aerosolized medicament for several days or weeks.

### Exemplary Nebulizers

In regard to the nebulizers (i.e., aerosol generators), they may be of the type, for example, where a vibratable member is vibrated at ultrasonic frequencies to produce liquid droplets (e.g., a vibrating mesh nebulizer). Some specific, non-limiting examples of technologies for producing fine liquid droplets is by supplying liquid to an aperture plate having a plurality of tapered apertures and vibrating the aperture plate to eject liquid droplets through the apertures. Such techniques are described generally in U.S. Pat. Nos. 5,164,740; 5,938,117; 5,586,550; 5,758,637, 6,014,970, and 6,085,740, the complete disclosures of which are incorporated by reference. However, it should be appreciated that the present invention is not limited for use only with such devices.

Referring now to FIG. 5, a vibrating mesh nebulizer 502 coupled to a T-piece 504 is shown. The nebulizer 502 may include a reservoir 506 that is orientated at a non-perpendicular angle to the T-piece 504. For example, the reservoir 506 may be formed at an angle between about 10° and about 75° with respect to an axis that is collinear with the base conduit of the T-piece 504. The reservoir 506 may have a cap 508 that can sealingly engage an opening in the reservoir 506 to contain a liquid medicament in the reservoir body 510. The cap 508 and top of the reservoir 506 may have conjugate threads or grooves that can be sealingly engaged to close the reservoir. Alternatively, the cap 508 may be made from an elastomeric material that can be elastomerically sealed or snapped into place around the opening in the reservoir 506. The reservoir 506 may be refilled by removing cap 508, adding liquid medicament to the reservoir body 510, and resealing the cap 508 on the reservoir 506. In the embodiment shown, about 4 mL of medicament may be stored in the reservoir body 510. In additional embodiments, the volume of medicament stored may range from about 1 mL to about 10 mL, and larger reservoirs may hold 10 mL or more of a medicament.

The nebulizer 502 may also include a power inlet 512 that can receive a plug 514 that supplies electric power to the nebulizer. Alternatively, the power inlet 512 may be replaced or supplemented by a power cord that terminates with a plug that can be plugged into a power source (not shown). The inlet 512 may also receive an electronic control signal that can control the timing and frequency which the nebulizer aerosolizes medicament from the reservoir 506.

FIG. 6 shows an exploded view of a vibrating mesh nebulizer 600 decoupled from the T-piece (not shown), according to an embodiment of the invention. An opening 602 in the nebulizer 600 that couples to the T-piece, or some other inlet in the PDDS, may include an aerosolization element 604 secured within the opening 602 by retaining element 606. In operation, medicament from the reservoir 608 passes through outlet 610 and is aerosolized by the aerosolization element 604. The aerosolized medicament may then drift or flow past retaining element 606 and into the PDDS. Alternative embodiments, not shown, may have the aerosolization element 604 permanently affixed, or integral to, the opening 602, and retaining element 606 may be absent.

The aerosolization element 604 may have a vibratable member that moves with respect to an aperture plate to aerosolized the liquid medicament. By utilizing an aerosol generator that produces aerosol by the electric powering of the vibratable member that causes the aperture plate to eject liquid at one face thereof, through its apertures, as a mist from the other face thereof, as generally described above (and as described generally in U.S. Pat. Nos. 5,164,740; 5,938,117; 5,586,550; 5,758,637, 6,085,740; and 6,235,177, the complete disclosures of which are, and have been above, incorporated herein by reference), the starting and stopping of aerosol generation may be controlled on the level of accuracy of microseconds or milliseconds, thus providing accurate dosing. The timing of aerosol generation can be done based solely on a predetermined timing within a breathing cycle, on timing in conjunction with the length of a prior breath or portions thereof, on other breathing characteristics, on particular medication being administered, or a combination of any of these criteria.

The aerosolization element may be constructed of a variety of materials, comprising metals, which may be formed to create a plurality of apertures as the element is formed, as described, for example, in U.S. Pat. No. 6,235,177; U.S. patent application Ser. Nos. 09/551,408 and 11/471,282; and US Patent Application Publication 2007/0023547, each of which is assigned to the present assignee and incorporated by reference herein in its erfirety. In one or more embodiments, the aerosolization element (also sometimes referred to as an aperture plate) comprises a Platinum Group metal or metals. In one or more embodiments, the aerosolization element comprises palladium, or a palladium alloy. In one or more embodiments, the aerosolization element is electroformed. Palladium is of particular usefulness in producing an electroformed, multi-apertured aerosolization element, as well as in operation thereof to aerosolize liquids. Other metals that can be used are palladium alloys, such as palladium nickel. If made as an alloy or combination, the palladium may comprise from about 60% or 70% or 75% or 80% or 85% or 90% or 95% or 99%, with the nickel comprising the remainder, or 40% or 30% or 25% or 20% or 15% or 10% or 5% or 1%. Other metals and materials may be used without departing from the scope of present invention.

In one or more embodiments, the aerosolization element is formed to have a plurality of tapered or conical-shaped apertures extending from a droplet—ejecting (or rear surface), to a liquid supply (or front surface), the plurality of apertures being tapered such that the liquid supply surface has the largest diameter, narrowing at the droplet-ejecting surface. In one or more embodiments, the apertures have an exit angle that is in the range from about 30° to about 60°, and a diameter that is in the range from about 1 micron to about 10 microns at the narrowest portion of the taper. In one or more embodiments, the aperture plate comprises a non-planar element, or dome-shaped element. In one or more embodiments, the non-planar, or dome-shaped portion of the aerosolization element comprises substantially all of the exposed area of the element, such as 75%, or 80% or 85% or 90% or 95% of the area of the element.

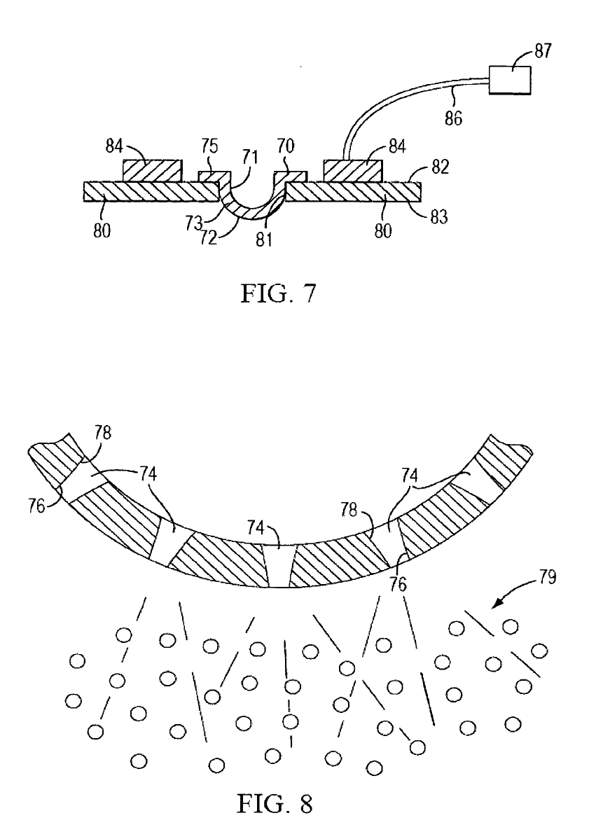

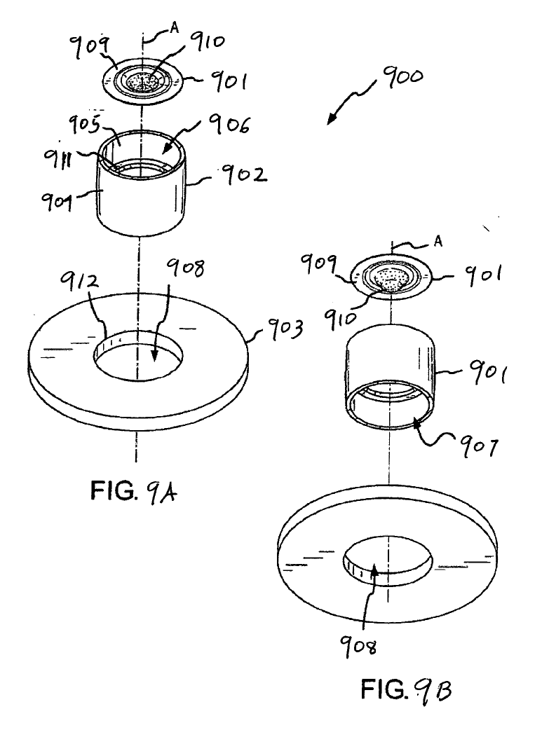

Referring now to FIGS. 7 and 8, an aerosolization element 70 may be configured to have a curvature, as in a dome shape, which may be spherical, parabolic or any other curvature. The aerosolization element may be formed to have a dome portion 73 over its majority, and this may be concentric with the center of the aerosolization element, thus leaving a portion of the aerosolization element that is a substantially planar peripheral ring portion 75. The aerosolization element has a first face 71, a second face 72. As shown in FIG. 8, the aerosolization element may also have a plurality of apertures 74 therethrough. The first face 71 may comprise the concave side of the dome portion 72 and the second face 72 may comprise the convex side of the dome portion 72 of the aerosolization element 70. The apertures may be tapered to have a wide portion 78 at the first face 71 and a narrow portion 76 at the second face 72 of the aerosolization element 70. Typically, a liquid will be placed at the first face of the aerosolization element, where it can be drawn into the wide portion 78 of the apertures 74 and emitted as an aerosolized mist or cloud 79 from the narrow portion 76 of the apertures 74 at the second face 72 of the aerosolization element 70.

The aerosolization element may be mounted on an aerosol actuator 80, which defines an aperture 81 therethrough. This may be done in such a manner that the dome portion of the aerosolization element protrudes through the aperture 81 of the aerosol actuator 80 and the substantially planar peripheral ring portion 74, on the second face 72 of the aerosolization element 70 abuts a first face 82 of the aerosol actuator 80. A vibratory element 84 may be provided, and may be mounted on the first face 82 of the aerosol actuator 80, or alternatively may be mounted on an opposing second face 83 of the aerosol actuator 80. The aerosolization element may be vibrated in such a manner as to draw liquid through the apertures 74 of the aerosolization element 70 from the first face to the second face, where the liquid is expelled from the apertures as a nebulized mist. The aerosolization element may be vibrated by a vibratory element 84, which may be a piezoelectric element. The vibratory element may be mounted to the aerosol actuator, such that vibration of the vibratory element may be mechanically transferred through the aerosol actuator to the aerosolization element. The vibratory element may be annular, and may surround the aperture of the aerosol actuator, for example, in a coaxial arrangement.

Embodiments of the invention include the aerosolization element, or the aerosol generator, comprising the aerosolization element 70, the aerosol actuator 80 and the vibratory element 86 may be replaced with an assembly that has apertures of a different size, such as a different exit diameter, to produce a mist having a different aerosol particle size. A circuitry 86 may provide power from a power source. The circuitry may include a switch that may be operable to vibrate the vibratory element and thus the aerosolization element, and aerosolization performed in this manner may be achieved within milliseconds of operation of the switch. The circuitry may include a controller 87, for example, a microprocessor that can provide power to the vibratory element 84 to produce aerosol from the aerosolization element 70 within milliseconds or fractions of milliseconds of a signal to do so. For example, aerosol production may begin within about 0.02 to about 50 milliseconds of such a signal and may stop within about 0.02 to about 50 milliseconds from the cessation of a first signal or a second signal either of which may act as a trigger to turn of aerosolization. Similarly, aerosol production may begin and end within about 0.02 milliseconds to about 20 milliseconds of such respective signaling. Likewise, aerosol production may begin and end within about 0.02 milliseconds to about 2 milliseconds of such respective signaling. Further, this manner of aerosolization provides full aerosolization with a substantially uniform particle size of low velocity mist 79 being produced effectively instantaneously with operation of the switch or element 84.

In one or more embodiments, an aerosol plume produced by the aerosolization element is low velocity. In one or more embodiments, the aerosol plume has an initial velocity (immediately downstream of the aerosol generator) of between about 0.5 and 8 meters per second (m/s). Typically, such a plume decelerates rapidly after generation.

In one or more embodiments the droplets 79 are of a respirable size, preferably between about 0.1 and 10 microns in size (which may be geometric diameter or mass median aerodynamic diameter). In one or more embodiments, the droplets 79 are greater than about 1 or 2 or 3 or 4 or 5 microns. In one or more embodiments, the droplets 79 are smaller than about 9 or 8 or 7 or 6 or 5 or 4 or 3 microns. In one or more embodiments, about 70% or more (by weight) of the droplets 79 have sizes from about 0.5 to about 7 microns, or about 0.5⁻to about 5 microns, or about 0.5 to about 3.5 microns, or about 1 to about 3 microns. In one or more embodiments, about 60% or more (by weight) of the droplets 79 have sizes from about 0.5 to about 7 microns, or about 1 to about 5 microns. In some embodiments the aerosol generator may generate a respirable fraction which is bimodal, that is a first fraction is between about 0.1 and 1 microns, and a second fraction is between about 1 and 5 microns.

In one or more embodiment, the aperture plate or aerosolization element is constructed so that a volume of liquid in the range from about 4 microliters to about 30 microliters may be aerosolized within a time that is less than about one second per about 1000 apertures. Further, each of the droplets may be produced such that a respirable fraction of droplets is greater than about 60% or 65% or 70% or 75% or 80% or 85% or 90% or more. A respirable fraction comprises the fraction which is within a respirable size range. In this way, a medicament may be aerosolized and then directly inhaled by a patient.

A transducer (not shown) may be used to sense the absence or presence of liquid in the reservoir, by sensing, for example, a difference between vibration characteristics of the aerosolization element, such as, for example, differences in frequency or amplitude, between wet vibration and substantially dry vibration. In this manner, the circuitry, may, for example by way of the microprocessor, turn the vibration off when there is essentially no more liquid to aerosolize, i.e., when the end of the dose has been achieved, thus minimizing operation of the aerosolization element in a dry state. Likewise, the switch may prevent vibration prior to delivery of a subsequent dose into the reservoir. An example of such a switch is shown in co-owned U.S. Pat. No. 6,546,927, the entire contents of which is hereby incorporated herein by reference.

The switch means, described above, may be operable by a pressure transducer, which may be positioned in the mouthpiece of the nebulizer. The pressure transducer may be in electrical communication with the circuitry, and a microprocessor may also be in electrical communication with the circuitry, and the microprocessor may interpret electrical signals from the pressure transducer, and may also operate the switch to begin aerosolization. In this manner, nebulization can begin substantially instantaneously with the inhalation of a user upon the mouthpiece. An example of such a sensor switch can be found in co-assigned PCT Publication No. WO2002/036181, the entire contents of which are hereby incorporated herein by reference.

A transducer (not shown) may be used to sense the absence or presence of liquid in the reservoir, by sensing, for example, a difference between vibration characteristics of the aerosolization element, such as, for example, differences in frequency or amplitude, between wet vibration and substantially dry vibration. In this manner, the circuitry, may, for example by way of the microprocessor, turn the vibration off when there is essentially no more liquid to aerosolize, i.e., when the end of the dose has been achieved, thus minimizing operation of the aperture plate 70 in a dry state. Likewise, the switch means may prevent vibration prior to delivery of a subsequent dose into the reservoir. An example of such a switch means or element is shown in co-assigned U.S. Pat. No. 6,546,927, the entire contents of which is hereby incorporated herein by reference.

In one or more embodiments, the aerosol generator controller may be configured to shut off the aerosol generator after one or more parameters, qualities or thresholds (as described above) are reached, such as shutting of the aerosol generator after a predetermined amount of nebulization time, and/or after a predetermined amount of liquid is aerosolized.

One or more embodiments of an aerosolization engine, or vibration system is shown in FIGS. 9A-B and 10, and designated by the general reference numeral 900. The system 900 comprises an aperture plate and alignment tube. A vibration system 900 comprises vibratable plate 901, tubular member 902 and piezoelectric ring 903. Tubular member 902 has an outer circumference 904 and an inner circumference 905, which together define a relatively thin cylindrical wall, and may preferably have a thickness in the range from about 0.1 mm to 0.5 mm. The hollow center (lumen) of tubular member 902 terminates in openings 906 and 907 at opposing ends thereof. Mounting structure 911 comprises a circular ridge that projects perpendicularly from inner circumference 905 into the lumen of tubular member 902 at a location, preferably a central location, between openings 906 and 907. Piezoelectric ring 903 comprises an annular disc of piezoelectric material having a center hole 908 with a circumference 912 approximately equal to the outer circumference 904 of tubular member 902. Vibratable plate 901 comprises circular outer flange 909 surrounding a thin circular vibratable center portion 910. In one or more embodiments, the plate 901, tubular member 902 and ring 903 are coaxial about a central axis AA.

In one method of making vibration system 900, metallic tubular member 902 may first be provided with mounting structure 911 by bonding a ridge of metal around inner circumference 905 at a location equidistant from ends 906 and 907. Vibratable plate 901 may then be concentrically disposed within the lumen of tubular member 902 with the lower surface of circular flange 909 positioned over the upper surface of mounting structure 911 and with the outer periphery of vibratable plate 901 abutting inner circumference 905. Outer flange 909 of vibratable plate 901 may be secured onto mounting structure 911 using a suitable joining procedure, e.g. a metallurgical process such as brazing, welding, soldering or the like, or a chemical bonding process such as adhesive bonding.

In one preferred embodiment, a brazing ring of a suitable corrosion-resistant brazing filler material, e.g. a mixture of gold and copper, may be placed between the upper surface of mounting structure 911 and outer flange 909 of vibratable plate 901. In one or more embodiments, the mixture comprises 60% or 65% or 70% or 75% or 80% gold, and correspondingly 40% or 35% or 30% or 25% or 20% copper. Other mixtures, alloys or combinations of metals may be used, such as silver, platinum, nickel and cobalt, in particular, nickel-cobalt. The entire assembly of tubular member 902, vibratable plate 901 and brazing ring may be held in place by a weight placed on top of vibratable plate 901. The assembly may be placed in an oven and heated to a temperature sufficient to melt the brazing and permanently join the surfaces together in a conventional brazing procedure. In other embodiments, vibratable plate 901 may be soldered onto mounting structure 911 using soldering materials, such as a tin/lead soldering material; however, this method may not be suitable if the assembly is to be exposed to acidic pharmaceutical preparations. In other embodiments, vibratable plate 901 may be secured onto mounting structure 911 by ultrasonic or laser welding.

Once vibratable plate 901 is secured across the lumen of tubular member 902, tubular member 902 may be positioned within center hole 908 of piezoelectric ring 903. In one embodiment, tubular member 902 may be placed in a fixture that holds tubular member 902 upright, and piezoelectric ring 903 may be slid lengthwise down tubular member 902 until piezoelectric ring 903 surrounds the outer circumference 904 at a location directly corresponding to the location of mounting structure 911 and vibratable plate 901 on inner circumference 905 of tubular member 902. Outer circumference 904 of tubular member 902 and circumference 912 of center hole 908 in piezoelectric ring 903 may then be bonded together, e.g. by depositing a suitable liquid adhesive around the juncture of circumference 904 and circumference 912 and curing the adhesive, e.g. with UV light. The adhesive used should be capable of efficiently transferring vibration from the piezoelectric ring 903 to tubular member 902. Although ideally the adhesive would have the modulus of elasticity (“Young's Modulus”) of the piezoelectric ring, i.e. about 60 GPa (Giga Pascal), to achieve the ultimate transfer of vibration, this is not possible for any adhesive. Most structural adhesives (such as epoxy) have a modulus of elasticity of plastic material, which may be about 2 GPa, and should be suitable for the present invention if cured to approximately that stiffness. As examples of suitable adhesives, mention may be made of various epoxy and anaerobic adhesives, such as commercially available UV-cured epoxy adhesives sold under the trademark Loctite.

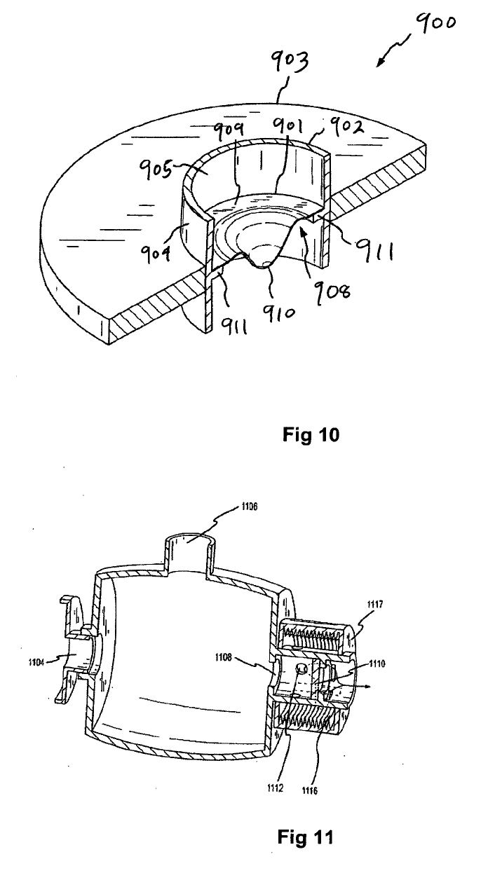

As previously described, piezoelectric ring 903 is configured to radially expand and contract when alternating electric fields are communicated to it via electric lines. refwerring also to FIG. 10, for example, piezoelectric ring 903 contracts radially towards its center opening when actuated by a first electric field. This radial contraction causes piezoelectric ring 903 to push inward along outer circumference 904 of tubular member 902 in the vicinity of mounting structure 911 and thereby pinch the wall of tubular member 902. The constriction of tubular member 902 causes flange 909 to also constrict radially and, as a result, the center portion 910 of vibratable plate 901 moves axially in direction A. When actuated by a second electric field, piezoelectric ring 903 expands radially away from its center opening, thereby releasing the inward pressure along circumference 904 of tubular member 902. This release of pressure allows flange 909 to expand radially, which causes center portion 910 of aperture plate 901 to move axially in direction A′ to its original position. Continually alternating the electric fields produces an oscillation (vibration) of center portion 910.

### Exemplary Aerosol Chamber

Embodiments of the invention may include an aerosolization chamber 1102 that can hold gas and aerosol mixtures for delivery to the patient's lungs. The chamber may be used in both on-ventilator and off-ventilator configurations. The expanded volume within the chamber reduces the surface area to volume ratio at the patient interface end of the system, which can increase the aerosol delivery efficiency. FIG. 11 shows an embodiment of such a chamber, with flow paths for gases and aerosols being inhaled and exhaled by a patient. The chamber 1102 may include a plurality of ports, including a gas inlet port 1104 that can receive gases from a ventilator, pump, and/or compressed gas source (e.g., a tank of compressed air, oxygen, etc.). The chamber 1102 may also include a second port 1106 that can receive a nebulizer (not shown), and a third port 1108 that can receive an endpiece (e.g., a mouthpiece, facemask, etc.).

Port 1108 may include a valve 1110 that can change the fluid flow path through the port 1108 depending on phase of a patient's breathing cycle. For example, during an inhalation phase, valve 1100 may be pushed away from the chamber 1102, channeling the gases and aerosols to flow around the ends of the valve into the endpiece (not shown), and ultimately into the patient's lungs. Then, during an exhalation phase, the valve 1110 is pushed by the patient's respiring gases to close port 1108, forcing the gases through openings 1112 and filters 1116 before exiting the filter housing 1117 into the surrounding atmosphere. The filter housing 1117 may include perforations that allow exhaled gases to exit and/or be constructed from gas permeable materials through which exhaled gas may diffuse.

As described herein, an aerosolization chamber may comprise a shaped body comprising a generally conical, or tapered, shape. In one or more embodiments the chamber shape is frusto-conical. In one or more embodiments the chamber comprises a conjoined double frustroconical shape, also known as a bifrustum. In one or more embodiments the chamber may have a ratio of maximum diameter to minimum diameter of about 5:4 to 2:1.

### Exemplary Medicaments

Examples of anti-gram-positive antibiotics or salts thereof include, but are not limited to, macrolides or salts thereof. Examples of macrolides or salts thereof include, but are not limited to, vancomycin, erythromycin, clarithromycin, azithromycin, dalbavancin, telavancin, salts thereof, and combinations thereof.

Vancomycin has been given intravenously (IV) for systemic therapy since it does not cross through the intestinal lining. It is a large hydrophilic molecule which partitions poorly across the gastrointestinal mucosa. The only indication for oral vancomycin therapy is in the treatment of pseudomembranous colitis, where it must be given orally to reach the site of infection in the colon.

There are factors which limit the drug's clinical utility when administered orally, or IV. Vancomycin must be administered in a dilute solution slowly, over at least 60 minutes (maximum rate of 10 mg/minute for doses >500 mg). This is due to the high incidence of pain and thrombophlebitis and to avoid an infusion reaction known as the red man syndrome or red neck syndrome.

Aerosolized delivery of vancomycin offers an attractive alternative to oral or intravenous delivery because it minimizes systemic exposure while delivering vancomycin directly to the site of infection. Aerosolized antibiotics have been administered as adjunctive therapy to mechanically-ventilated patients with deep lung infections—specifically nosocomial pneumonia and tracheobronchitis. Efforts to improve therapies of inhalation antibiotics have been hampered by the low efficiency of pulmonary drug delivery with conventional nebulizers connected to ventilator circuits.

Embodiments of the invention contemplate a variety of medicaments that can be aerosolized and delivered to a patient's lungs. These medicaments may include antibiotics such as glycopeptides, aminoglycosides, β-lactams, and quinolines, among others. The glycopeptides may include, for example, vancomycin, teicoplanin, ramoplanin, and decaplanin, dalbavancin and telavancin among other glycopeptides. The aminoglycosides may include amikacin, gentamycin, kanamycin, streptomycin, neomycin, netilmicin, and tobramycin, among other aminoglycosides. Other medicaments may also be used, including anti-oxidants, bronchodilators, corticosteroids, leukotrienes, prostacyclins, protease inhibitors, and surfactants, among other medicaments. Table 1 lists classes of medicaments and some of the aliments they may be used to treat in their aerosolized state.

TABLE 1
Classes of Aerosolizable Medicaments

| | | | Duration of |
|---|---|---|---|
| Medicament Class | Aliments Treated | Dosing* | Treatment* |
| Anti-oxidants | RDS, Prevention of BPD, ALI, | 1-4 per day | Duration of |
| | ARDS | | ventilation |
| Bronchodilators | Asthma, COPD, ARDS, RDS | 1-4 per day | As needed |
| Corticosteroids | Asthma, COPD, BPD | 1-2 per day | Duration of |
| | | | ventilation |
| Leukotrienes or | Immunodeficiency, COPD, | 1-4 per day | 5-14 days |
| related agonists | Treatment/prevention of | | |
| | pneumonia or RSV infection | | |
| Prostacyclin or | PPHN, Secondary pulmonary | Continuous | TBD |
| related analogues | hypertension, Post-cardiac | | |
| | surgery, ARDS | | |
| Protease inhibitors | AECOPD, ARDS, RDS, BPD | 1-2 per day | 5-14 days |
| Surfactants | RDS, Prevention of BPD, | 1-2 per day | TBD |
| | ARDS | | |
| Oligopeptides | Asthma, COPD, ARDS, RDS | 1-2 per day | TBD |
| siRNA | Asthma, COPD, ARDS, RDS | 1-2 per day | TBD |

*this Table is only exemplary, and not intended to limit the diseases or conditions treated, or the methods of administering these medicaments, to any of the parameters listed.
AECOPD: acute exacerbation of COPD; ALI: Acute lung injury; ARDS: Acute respiratory distress syndrome; BPD: Bronchopulmonary dysplasia; COPD: chronic obstructive pulmonary disease; PPHN: persistent pulmonary hypertension; RDS: Respiratory distress syndrome (also known as infant respiratory distress syndrome); RSV: Respiratory syncytial virus.

Vancomycin is a tricyclic glycopeptide antibiotic produced by certain strains of Amycolatopasis orientalis, previously designated Streptomyces orientalis (formerly Nocardia orentalis). Vancomycin hydrochloride is a mixture of related substances consisting principally of the monohydrochloride of vancomycin B. As with all glycopeptide antibiotics, vancomycin hydrochloride contains a central core heptapeptide.

Vancomycin inhibits cell wall biosynthesis in susceptible microorganisms and alters bacterial cell-membrane permeability and RNA synthesis. Vancomycin is active against several gram-positive pathogens, including both methicillin-resistant (or sensitive) Staphylococcus aureus (MRSA), Clostridium species, and Pseudomonas species, including aeruginosa.

Vancomycin is both bactericidal (capable of killing bacteria) and bacteriostatic (capable of inhibiting growth and reproduction of bacteria without killing). Vancomycin's primary mechanism of action (inhibition of cell wall synthesis) is bactericidal and requires actively growing and dividing bacteria. Its secondary mechanisms (alteration of membrane permeability and inhibition of RNA synthesis) are both bactericidal and bacteriostatic. These mechanisms are thought to cause a small degree of concentration-dependent killing, but their main effects are to inhibit growth and reproduction.

In intubated, mechanically-ventilated patients with gram-positive pneumonia, as well as in freely-breathing patients, inhaled delivery is expected to provide higher doses of antibiotic to the target site (i.e., the lung) than can be achieved with IV administration while resulting in lower blood levels than IV infusion.

If lower systemic levels are achieved, the risk of systemically induced toxicities may be reduced. In addition, previous clinical experience indicates that the risk of antibiotic resistance is low when antibiotics are administered in aerosol form and that adverse pulmonary effects are extremely rare when preservative-free formulations of antibiotics are administered as aerosols

A recent randomized study confirms that aerosolized vancomycin treatment (n=14) for gram-positive bacteria and/or gentamicin-sulfate for gram-negative ventilator-associated tracheobronchitis (VAT) had reduced signs of respiratory infection: reduced Centers for Disease Control National Nosocomial Infection Survey (CDC-NNIS) diagnostic criteria for ventilator-associated pneumonia (VAP) 35.7% to 73.6% versus placebo 75.0% to 78.6%, reduced clinical pulmonary infection score CPIS, lower WBC at Day 14, reduced bacterial resistance, reduced use of systemic antibiotics, and increased ventilator weaning (all P values≦0.05). It has been shown that aerosolized vancomycin in mechanically ventilated patients (n=10) with high suspicion for MRSA respiratory infection significantly increases sputum vancomycin concentrations compared to vancomycin administered systemically (Zarrilli et al., 2008, abstract submitted ATS). The foregoing study thus corroborates and supports the conclusions of the inventors herein that aerosolized delivery of glycopeptides, such as vancomycin, directly to the pulmonary system, affords high (therapeutic) lung levels and low serum levels.

It is thought that adjunctive inhalational vancomycin offers an efficacy advantage over standard of care for intubated patients who have pulmonary conditions such as MRSA. Tissue penetration of parenterally administered vancomycin is poor with levels in lung epithelial lining fluid amounting to only ˜14% of those seen in serum. To compound matters, upward “MIC creep” has been described for vancomycin in recent years. Furthermore, the vancomycin breakpoint has recently been lowered by the Clinical and Laboratory Standards Institute (CLSI) to 2 μg/mL. Thus, S. aureus appears to be evolving increasing resistance to vancomycin at roughly the same time there is mounting appreciation that isolates of S. aureus once classified as sensitive may truly show evidence of diminished susceptibility to this agent. Furthermore, heteroresistance to vancomycin (i.e., the existence of resistant or less sensitive sub-populations) has been increasingly recognized.

The achievement of vancomycin levels in respiratory secretions that are high multiples of the MIC values for most hospital-acquired organisms has the potential to reduce total antibiotic days in nosocomial pneumonia patients, a strong determinant of the risk of antibiotic resistance.

The achievement of vancomycin levels in respiratory secretions that are high multiples of the MIC values for most hospital-acquired organisms also may help avoid exposure to systemic antibiotics, to strengthen short-course therapy for nosocomial pneumonia, reducing risk of relapse, and to hasten the resolution of nosocomial pneumonia, resulting in reduced mechanical ventilation and ICU days.

In one or more embodiments, the medicament which is aerosolized (and the aerosolized medicament) comprises vancomycin which is preservative free.

In one or more embodiments, the medicament which may be aerosolized comprises vancomycin having one or more of the characteristics of: a pH between about 2.5 and 4.5, a viscosity of between about 1.3 and 1.5 cSt, a surface tension of between about 50 and 60 mN/m, a density of about 0.99 to 1.06 g/mL, and an osmolality of about 100 to 300 mMol/kg. In one or more embodiments, the medicament which may be aerosolized comprises vancomycin having one or more of the characteristics of: a pH about 3.0 and 4.0, a viscosity of between about 1.4 and 1.45 cSt, a surface tension of between about 52 and 58 mN/m, a density of about 0.99 to 1.06 g/mL, and an osmolality of about 130 to 250 mMol/kg. In one or more embodiments, an osmolality of the liquid vancomycin to be aerosolized is close to, or at, an isotonic level for the target cell.

A useful measure of antibiotic activity is the minimum inhibitory concentration (MIC). The MIC is the lowest concentration of an antibiotic that completely inhibits the growth of a microorganism in vitro. While the MIC is a good indicator of the potency of an antibiotic, it indicates nothing about the time course of antimicrobial activity.

Pharmacokinetics (PK) parameters quantify the serum level time course of an antibiotic. Three pharmacokinetic parameters that are used to evaluate antibiotic efficacy (as illustrated in FIG. 23) are (1) The peak serum level (Cₘₐₓ); (2) The trough level (Cₘᵢₙ); and (3) The Area Under the serum concentration time Curve (AUC). While these parameters quantify the serum level time course, they do not describe the killing activity of an antibiotic.

Integrating the PK parameters with the MIC gives us three pharmacokinetic to pharmacodynamic (PK/PD) parameters which quantify the activity of an antibiotic: (1) The Peak/MIC ratio; (2) The T>MIC; and (3) The 24 h-AUC/MIC ratio. The Peak/MIC ratio is simply the Cₘₐₓ(peak) divided by the MIC. The T>MIC (time above MIC) is the percentage of a dosage interval in which the serum level exceeds the MIC. The 24 h-AUC/MIC ratio is determined by dividing the 24-hour-AUC by the MIC.

The three pharmacodyamic properties of antibiotics that best describe killing activity are time-dependence, concentration-dependence, and persistent effects. The rate of killing is determined by either the length of time necessary to kill (time-dependent), or the effect of increasing concentrations (concentration-dependent). Persistent effects include the Post-Antibiotic Effect (PAE). PAE is the persistant suppression of bacterial growth following antibiotic exposure.

Using these parameters, antibiotics may be divided into 3 categories:

| | | | |
|---|---|---|---|
| | | Goal of | |
| Pattern of Activity | Antibiotics | Therapy* | PK/PD Parameter |

Type I

| | | | |
|---|---|---|---|
| Concentration- | Aminoglycosides | Maximize | 24 h-AUC/MIC |
| dependent killing | Daptomycin | concen- | Peak/MIC |
| and Prolonged | Fluoroquinolones | trations | |
| persistent effects | Ketolides | | |

Type II

| | | | |
|---|---|---|---|
| Time-dependent | Carbapenems | Maximize | T > MIC |
| killing and Minimal | Cephalosporins | duration | |
| persistent effects | Erythromycin | of exposure | |
| | Linezolid | | |
| | Penicillins | | |

Type III

| | | | |
|---|---|---|---|
| Time-dependent | Azithromycin | Maximize | 24 h-AUC/MIC |
| killing and | Clindamycin | amount | |
| Moderate to | Oxazolidinones | of drug | |
| prolonged | Tetracyclines | | |
| persistent effects. | Vancomycin | | |

*this Table is only exemplary, and not intended to limit the methods of administering these antibiotics only to the “Goals of Therapy” listed in Column 3.

For Type I antibiotics (AG's, fluoroquinolones, daptomycin and the ketolides), the ideal dosing regimen would maximize concentration, because the higher the concentration, the more extensive and the faster is the degree of killing. Therefore, the 24 h-AUC/MIC ratio, and the Peak/MIC ratio are important predictors of antibiotic efficacy. For aminoglycosides, it is best to have a Peak/MIC ratio of at least 8-10 to prevent resistence. For fluoroquinolones versus gram negative bacteria, the optimal 24 h-AUC/MIC ratio is approximately 12 versus gram positives, and 40 may be optimal in some circumstances.

Type II antibiotics (beta-lactams, clindamycin, erythromcyin, and linezolid) demonstrate the complete opposite properties. The ideal dosing regimen for these antibiotics maximizes the duration of exposure. The T>MIC is the parameter that best correlates with efficacy. For beta-lactams and erythromycin, maximum killing is seen when the time above MIC is at least 70% of the dosing interval.

Type III antibiotics, including vancomycin as well as tetracyclines, azithromycin, and the dalfopristin-quinupristin combination, have mixed properties. They have time-dependent killing and moderate persistent effects. The ideal dosing regimen for these antibiotics maximizes the amount of drug received. Therefore, the 24 h-AUC/MIC ratio is the parameter that correlates with efficacy. For vancomycin administered conventionally (such as intravenously and/or orally), a 24 h-AUC/MIC ratio of at least 125 is considered necessary.

Embodiments of the invention include methods of administering aerosolized vancomycin to a patient that reflects the antibiotic's Type III classification. The methods herein include administering aerosolized vancomycin such that a ratio of an amount of the antibiotic delivered to the patient in a 24-hour period to a minimum inhibitory amount for the same period is about 2 or 4 or 6 or 8 or 10 or more. In one or more embodiments, a goal of these administration methods is to increase the amount of vancomycin delivered instead of maximizing the peak concentration of the antibiotic in the patient or maximizing the duration of exposure. The methods may also include delivering the aerosolized vancomycin in an intermittent (e.g., phasic) or continuous manner.

Glycopeptide (such as vancomycin) concentrations in the trachea, when administered according to one or more embodiments of devices, apparatus and/or methods of the present invention, will be high following administration, and will diminish with time. In one or more embodiments, administration in accordance with the systems, devices and methods herein will result in therapeutically efficacious (high) glycopeptide local concentrations (i.e. in the trachea and/or lungs and/or pulmonary system), as measured in tracheal aspirates (TA), and/or in epithelial lining fluid (ELF) as recovered by bronchoalveolar lavage (BAL) and/or in sputum.

Vancomycin concentrations in the ELF may vary according to the zone sampled, but are expected to always be high and to exceed the vancomycin Minimum Inhibitory Concentration (MIC) for microorganisms usually responsible for Gram-positive lung infections, such as pneumonia, regardless of the daily dose received. Aerosolized vancomycin thus administered in accordance with one or more devices, apparatus and methods of the present invention is expected to be well tolerated.

### Exemplary Indications

One or more embodiments of the invention relate to compositions and methods for treating bacterial infections. One or more embodiments comprise compositions and methods for the treatment of Cystic Fibrosis (CF). One or more embodiments comprise compositions and methods for the treatment of Pneumonias, such as VAP. HAP or CAP.

Persons with CF typically suffer from chronic endobronchial infections, sinusitis, and malabsorption due to pancreatic insufficiency, increased salt loss in sweat, obstructive hepatobiliary disease, and reduced fertility. Respiratory disease is a major cause of morbidity and accounts for 90% of mortality in persons with CF.

CF patients suffer from thickened mucus caused by perturbed epithelial ion transport that impairs lung host defenses, resulting in increased susceptibility to early endobronchial infections with Staphylococcus aureus, Haemophilus influenzae, and Pseudomonas aeruginosa. By adolescence, a majority of persons with CF have P. aeruginosa present in their sputum. A link between acquisition of chronic endobronchial P. aeruginosa infection, lung inflammation, loss of lung function, and ultimate death is suggested by significantly decreased survival associated with chronic P. aeruginosa infections.

One or more embodiments of the invention thus comprises early treatment in of Staphylococcus infections in CF patients. One or more embodiments of the invention thus comprises treatment in of Pseudomonas infections in CF patients.

One or more embodiments of the invention relate to compositions and methods for treating suppurative diseases, for example pleura, empyema thoracis, lung abscess, and bronchiectasis, bronchiolitis and tuberculosis. Embodiments of the invention comprise systems, apparatus and methods to administer glycopeptides, especially vancomycin, to maximize and/or optimize its pharmacodynamic properties, comprising time-dependent killing and post-antibiotic effects. Thus high concentrations of aerosolized vancomycin (and/or other glycopeptides) can advantageously be delivered directly to the pulmonary system of a patient by nebulization using a vibrating mesh nebulizer. In one or more embodiments, a concentration of 20 or 30 or 40 or 50 or 60 or 70 or 80 or 90 or 100 or 110 or 120 or 130 or 140 or 150 or 160 or 170 or 180 or 190 or 200 or more mg/mL of glycopeptide (such as vancomycin) is delivered. In one or more embodiments, an aerosolized glycopeptide (such as vancomycin) flow rate is about 0.10 or 0.20 or 0.30 or 0.40 or 0.50 or 0.60 or 0.70 or 0.80 or 0.90 or 1.0 or more Liters per minute (Lpm).

In one or more embodiments, an amount of aerosolized glycopeptide (such as vancomycin) delivered to the lungs and/or pulmonary system is at least a therapeutic dose, such as about 40 or 50 or 100 or 150 or 200 or 250 or 300 or 350 or 400 or 450 or 500 or 550 or 600 milligrams (mg) or more of vancomycin or vancomycin hydrochloride. In one or more embodiments, a delivery time for a therapeutic dose of an aerosolized glycopeptide (such as vancomycin) is less than about 18 minutes or 15 minutes or 12 minutes or 10 minutes or 5 minutes or 4 minutes or 3 minutes or 2 minutes. In one or more embodiments, a nebulization rate may be about 0.1 or 0.2 or 0.3 or 0.4 or 0.5 mL/minute. In one or more embodiments, a therapeutic dose may be 0.5 or 1.0 or 1.5 or 2.0 or 2.5 or 3.0 or 3.5 or 4.0 or 4.5 or 5.0 or more milliliters of glycopeptide solution. In one or more embodiments, a does, such as a therapeutic dose, is given once daily or twice daily or three times daily or more.

In one or more embodiments, aerosolized glycopeptide (such as vancomycin) delivered to the lungs and/or pulmonary system according to the present invention is expected to reduce the incidence and/or severity and/or duration of VAP (for patients on ventilators), and/or reduce the incidence and/or severity and/or duration of CAP and/or reduce the incidence and/or severity and/or duration of HAP.

In one or more embodiments, aerosolized glycopeptide (such as vancomycin) delivered to the lungs and/or pulmonary system of patients requiring mechanical ventilation according to the present invention is expected to reduce the duration of such mechanical ventilation (apart from the underlying causation for mechanical ventilation, e.g. trauma), such as by 10% or 20% or 30% or 40% or 50% or 60% or 70% or more.

In one or more embodiments, aerosolized glycopeptide (such as vancomycin) delivered to the lungs and/or pulmonary system according to the present invention is expected to reduce the need for systemic antibiotic.

In one or more embodiments, aerosolized glycopeptide (such as vancomycin) delivered to the lungs and/or pulmonary system according to the present invention is expected to reduce the emergence of antibiotic-resistant bacteria strains, such as by 10% or 20% or 30% or 40% or 50% or 60% or 70% or more.

The dose-response to aerosolized vancomycin, administered in accordance with one or more embodiments of devices, apparatus and/or methods of the present invention was evaluated, as was the ability to deliver a predefined target multiple of an MIC for Gram-positive bacteria causing pneumonia, including, Staph species, in the tracheal aspirates (TA). Thus, one or more embodiments of devices, apparatus and/or methods of the present invention is used to deliver four times a reference MIC value for Gram-positive, pneumonia-causing bacteria, which value was determined to be 32 μg/mL locally in TA. Thus the desired multiple is 128 μg/mL.

### Exemplary Phasic Delivery Methods

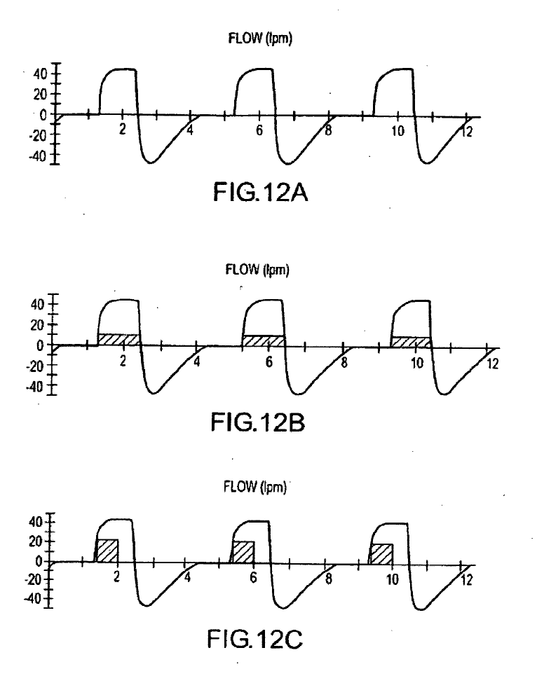

FIGS. 12A-C show graphs of various modes of aerosolization over the course of breathing cycles. FIG. 12A shows a continuous aerosolization mode where aerosolized medicament is generated a constant rate throughout the breathing cycle. Continuous (i.e., aphasic) generation modes typically have about 10% to about 15% aerosol delivery efficiency. FIG. 12B shows a phasic delivery mode where aerosolized medicament is administered for substantially all of the inhalation phase of the breathing cycle. These modes typically have about 15% to about 25% efficiency. FIG. 12C shows another phasic delivery mode where the aerosolized medicament is administered during a predetermined portion of the inhalation phase beginning, for example, at the onset of inhalation. It has been discovered that these modes typically have delivery efficiencies between about 60% to about 80%, by weight, of the total amount of medicament that is aerosolized.

Embodiments of the invention take advantage of this discovery by controlling delivery to a predetermined percentage of the breathing cycle, such as a predetermined percentage of the inhalation phase of the breathing cycle, to provide greater delivery efficiency than either continuous delivery or delivery during the entire inhalation phase. Embodiments of the invention also take advantage of the surprising discovery that the percentage of increase in efficiency in delivery for such a predetermined portion of the inhalation phase over delivery during the entire inhalation phase is itself greater than the increase in efficiency of delivery during the inhalation phase compared to aphasic administration of the aerosol.

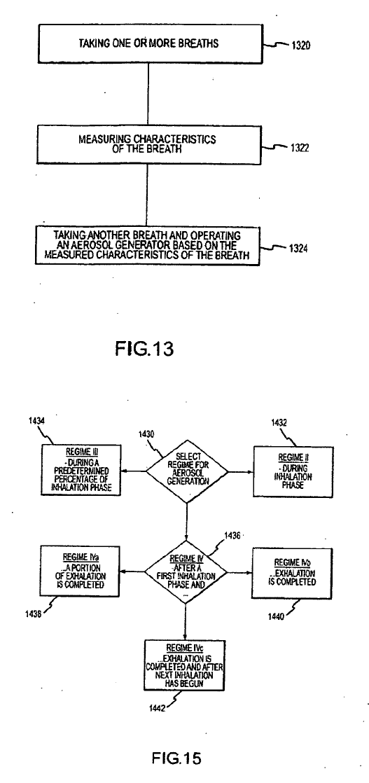

Phasic delivery methods may include measuring the characteristics of a patient's inhaled breath, typically a tidal breath, and using the measurements to control the operation of the aerosol generator. FIG. 13 provides a simplified flowchart that illustrates some of the steps for phasic delivery of an aerosolized medicament according to embodiments of the invention. Phasic delivery methods may include having a patient can take one or more breaths 1320, and measuring the characteristics of the breath 1322. The breathing characteristics that can be measured include, but are not limited to, a breathing pattern, peak inspiratory flow rate, breathing rate, exhalation parameters, regularity of breathing, tidal volume, and the like and can estimate a user's tidal volume based on such information.

The user can take another tidal breath and the aerosol generator can be operated based on the measured characteristics of the tidal breath 1324. It should be appreciated however, that instead of a tidal breath, the person can take other types of breath. Alternatively, the controller may base the timing of operation of the aerosol generator so that aerosol is generated at specific time periods within a breathing cycle. For example, the controller may operate the aerosol generator for the first 50 percent of inspiration. Alternatively, the controller may operate the aerosol generator to generate aerosol after a portion of inhalation has taken place and to cease producing aerosol after another portion of inhalation has taken place. For example, the controller may cause aerosol to be generated beginning after 20% of the inspiration has taken place and cause aerosol production to cease after 70% of inspiration has taken place. The controller may cause aerosol production to start after, for example, after 90% of exhalation has taken place and, for example, cause aerosol production to stop after 30% of the following inspiration has taken place. By controlling the specific timing within the breathing cycle that aerosolized medication is provided into the breathing circuit, greater efficiency of drug administration can be achieved.

Since some of the pharmaceuticals to be aerosolized may be more effective when delivered near the beginning of a patient's breathing cycle, while other pharmaceuticals may be more effective when delivered near the end of the patient's breathing cycle, the timing of the aerosol generation depends on the type of medicament delivered. If it is known what type of medication or drug is being delivered, the controller can select the best time during the patient's breathing cycle to deliver the aerosol, based upon a predetermined regimen for that drug that is stored in memory. As an additional benefit, an estimate of the patient's age and/or distress can be made, for example, by measuring the tidal volume and breathing rate. Such measurements can influence the efficiency requirements of the dose per breath. These or other variables can be used in establishing various regimes for aerosol delivery, in particular delivery into the breathing circuit of a ventilator. These regimes can be stored in memory and then accessed by the controller as appropriate for a given patient condition.

For example, for a bronchodilator the best time to delivery may be half way through the inhalation phase of a breath when impaction would be reduced since inhalation flows are reducing. For steroids, it may be best to deliver towards the end of the inhalation phase of a breath. For antibiotics, it may be best to slightly pre-load, e.g., deliver aerosol during the exhalation phase, or deliver right at the start of the breath. For example, antibiotics may be delivered at the beginning of a ventilator provided inhalation, and the aerosol delivery may stop after a predetermined percentage of the inhalation has been provided.

One class of antibiotics that may be administered in accordance with the present invention is the class known as the glycopeptide (including lipoglycopeptide) class of antibiotics. This class of antibiotics has typically been administered intravenously, however, such delivery can sometimes have unwanted side effects, which may be systemic. Embodiments of the invention provide for the administration of antibiotics, such as glycopeptides including vancomycin by delivering them in aerosolized form into the breathing circuit of a patient on a ventilator. In this manner, vancomycin can be used to treat pulmonary infection conditions that typically arise when patients are mechanically ventilated, and the vancomycin, or other glycopeptide or other antibiotic, can be delivered directly to the target of treatment, the pulmonary tract, avoiding side effects that may otherwise arise from intravenous administration. Further, because of the great cost of such drugs, far greater efficiency is achieved through this pulmonary delivery. As noted above with reference to FIG. 12C, delivery of aerosol during a beginning percentage of the inhalation phase of a breathing cycle may yield up between about 60% and about 80% efficiency, a significantly higher efficacy than continuous aerosolization, or aerosolization for an entire inhalation phase of an inhalation cycle.

For mechanically-ventilated patients, the PDDS control module operates in optimized phasic mode, generating aerosol during a specific percentage of the ventilator inspiratory cycle. The percent of inspiratory time is preset for each device so that aerosol is generated during the first 75% of inspiration. The device generates aerosol with positive pressure breaths generated by the ventilator. For patients who have been taken off the ventilator before the course of inhaled medicament (e.g. glycopeptide) is complete, the PDDS can be modified for handheld use such that it continues to provide high efficiency drug delivery via a mouthpiece (and mouth seal if needed). The presence of a filter minimizes environmental aerosol exposure. During handheld use, the PDDS control module operates by generating aerosol continuously.

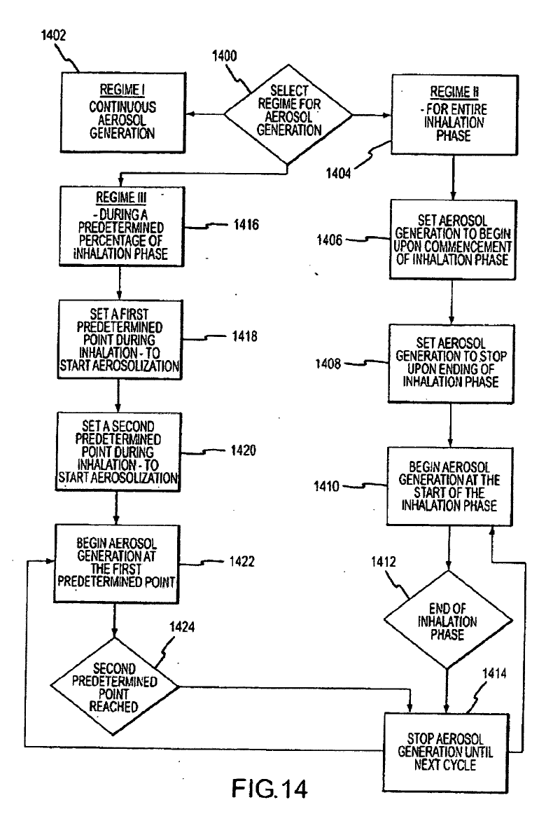

Embodiments of the invention provide for conducting various regimes of aerosolization, depending on the situation. For example, in FIG. 14, a selection between a first, second and third regime is shown. A regime may be selected manually or automatically, for example, through the application of an algorithm that selects an operation program based on information that is either input or stored. For manual selection, a user may operate a mechanical switch to select a regime, or may enter such a selection into an electronic input device, such as a keyboard. Alternatively, the controller may automatically choose a regimen, as described above, by matching a drug code on a drug nebule with a library of drug-regimen combinations. It should be noted that in FIGS. 14-17, schematic flow charts of operation sequence algorithms are depicted. Although items therein will be referred to as steps for ease of discussion, they refer more broadly herein to states of operations or modalities in which a system may exist or cycle through. Steps depicted in a rectangle are essentially states of operation, actions or modalities. Steps depicted in diamonds indicate either a selection or the continuance of the previous state of operation, action or modality until a predetermined condition is satisfied. Two successive diamonds refer to satisfaction of a first condition and of a second condition respectively, the second of which may be a subset of the first.)

In step 1400, a choice is made to follow a particular regime. In this case, regime I is a regime in which aerosol is generated continuously (step 1402). Regime ll provides aerosol generation during the inhalation phase only (step 1404). In this case, in step 1406, aerosol generation is set to start at the start of the inhalation phase and, in step 1408, aerosol generation is set to stop when the inhalation phase stops. In step 1410, aerosol generation begins at the start of the inhalation phase. In step 1412, when the inhalation phase ends, aerosol generation stops (step 1414).

Regime III provides for inhalation during a predetermined percentage of the inhalation phase (step 1416). A predetermined percentage of an inhalation (or exhalation) phase may be based on a measured time from a discrete point in the ventilator cycle, such as the instantaneous commencement of inspiratory air generation by the ventilator. Alternatively, such predetermined percentage may be based on the time interval between successive discrete points in the ventilator, such as successive commencements of successive inhalation air generation by the ventilator. Alternatively, such percentages may be based upon air pressure in the ventilator circuit, or any other parameter. With respect to Regime III, in this case, in step 1418, a first predetermined point is set to correspond with the completion of a first predetermined percent of the inhalation. In step 1420, a second predetermined point is set to correspond to a second predetermined percent of inhalation percent being completed. For example, as described above, the first predetermined point may correspond to 20% of the inhalation phase being completed, and the second predetermined point may correspond to a point at which 70% of that same inhalation has taken place. In step 1422, aerosol generation begins at the first predetermined point in the inhalation phase. In step 1424, when the second predetermined point is reached, the controller carries out step 1414 and stops the aerosol generation.

Similarly, as noted above, other regimes may be followed, for example, in which aerosol generation begins during the inhalation phase and ends during the exhalation phase, or begins during exhalation and ends during that exhalation, or begins during exhalation and ends in the subsequent breath cycle, for example, at a predetermined point in the subsequent inhalation phase. Accordingly, turning to FIG. 15, a selection may be made, at step 1430, between regimes II (step 1432) and III (step 1434) as described above, and another regime, regime IV (steps 1436-1442), which is also available for selection. In regime IV, aerosol generation may begin at a first predetermined point (step 1436), and this first predetermined point may be after a predetermined percentage of the inhalation phase has taken place, or it may be a predetermined point after the inhalation phase has been completed. For example, this point may be a predetermined point after a predetermined percent of the exhalation phase has taken place, or may be a predetermined point prior to the start of the subsequent inhalation phase. Aerosol generation may stop during exhalation (regime IVa, step 1438), at the completion of exhalation (regime IVb, step 1440), or aerosol generation may continue into the next breath cycle (regime IVc, step 1442), and stop, for example, after a predetermined point during the subsequent inhalation phase.

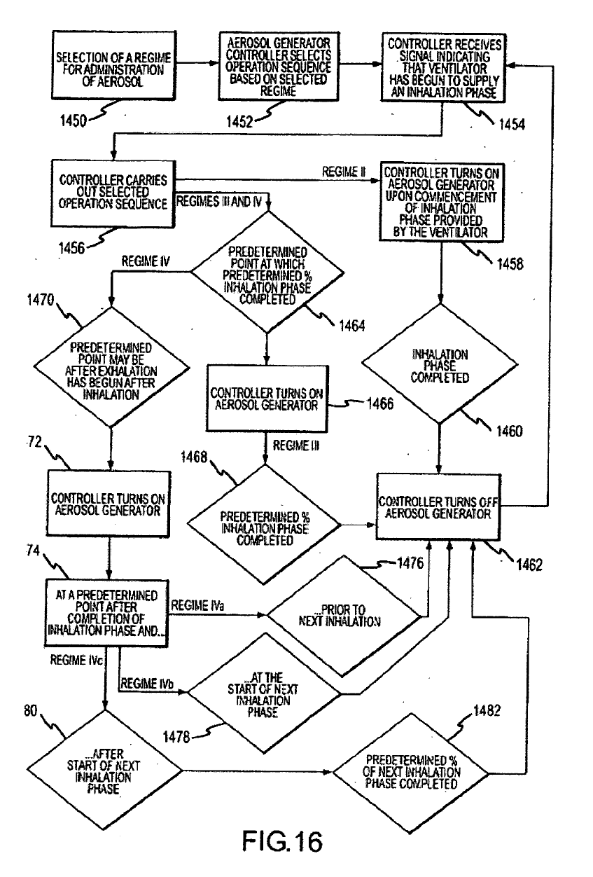

In this example, with the controller having a selection choice between operation sequences corresponding to regimes II, III and IV, schematic representation of the operation sequences are shown in FIG. 16. In step 1450, a regime is selected. In step 1452, the aerosol generator controller selects an operation sequence based on selected regime. In step 1454, the controller receives a signal indicating that ventilator has begun to supply an inhalation phase. The signal, as described above, may be a signal provided directly by the ventilator. Alternatively, the signal may be provided by a sensor, and such sensor may sense the commencement of an inhalation phase provided by the ventilator, as described above, by sensing a pressure change in the breathing circuit. In step 1456, the controller carries out selected operation sequence. In the case of regime II (step 1458), the controller turns on aerosol generator upon commencement of inhalation phase provided by the ventilator. The controller continues to operate the aerosol generator until a point at which the inhalation phase completed (step 1460). In step 1462, controller turns off aerosol generator.

In the case of regime III, the controller does not take any action to begin aerosol generation, until a predetermined point in the inhalation phase, corresponding to a percentage of the inhalation phase being completed (step 1464). In step 1466, at a predetermined point in the inhalation phase, the controller turns on aerosol generator. In step 1468, aerosol generation continues until a second predetermined point inhalation phase, corresponding to a second percentage point of completion of the inhalation phase. At this point, the controller carries out step 1462 and turns off aerosol generator. With respect to regime IV, aerosol generation begins after a predetermined point of completion of the inhalation phase (step 1464) and this point may be predetermined to occur after the inhalation phase has been completed and the exhalation phase has begun (step 1470). In step 1472, the controller turns the aerosol generator on to begin aerosolization. Variations can be made as to the point at which the aerosol generation is turned off. If it is desired that aerosol generation be completed before the completion of the exhalation phase (regime IVa), then aerosol generation may continue until a predetermined point prior to the subsequent inhalation (step 1476). Alternatively, it may be desirable to continue aerosolization until the end of exhalation, which may correspond to the point of commencement of the subsequent inhalation, as in regime IVb (step 1478). Alternatively, it may be desired to follow a regimen such as regime IVc, where aerosol generation continues through into the subsequent breath cycle (step 1480), until, for example, a predetermined percent of the subsequent inhalation phase has been completed (step 1482). In these regimes, aerosolization will continue until the satisfaction of these conditions (step 1476 for regime IVa, step 1478 for regime IVb or step 1482 for regime IVc), at which point the controller carries out step 1462 and stops the aerosol generator. The process may continue with the next signal indicating that the ventilator has begun to provide an inhalation phase, step 1454.

Further, the choice of which operating sequence to follow may rely at least in part on the identity of a drug to be administered, which information can be considered by the controller as described above. In addition, it should be appreciated that modifications may be made to these examples without departing from the present invention. For example, a system may be configured, or a method may be carried out, to be able to select more than three initial regimes to follow. For example, regimes I, II, III and IV as described above may be simultaneously selectable. Further, various steps may be altered; for example, some steps may not be discrete steps. Thus, step 1456 may not be a discrete step but rather the following of an operation sequence according to a selected regime. Similarly, the order of the steps may be changed, such as the controller may select an operating sequence (step 1452) after receiving a signal that the ventilator has commenced to provide an inhalation phase (step 1454). Steps may also be combined, such as, for example, in regime IV steps 1464 and 1470 may be combined as a single step, as these two steps represent successive criteria for the determining a single first predetermined point has been met. Likewise, step 1474 may be combined with steps 1476, 1478 or 1480, as step 1474 is the predicate for the condition test specified in each of the other successive tests, steps 1476, 1478 or 1480. The algorithm examples may be altered to form other operating sequences. For example, an operating sequence may call for the controller to start aerosol generation at the start of the inhalation cycle provided by the nebulizer, as in regime II, at step 1458, and turn off the aerosol generator at a point at which a predetermined percentage of the inhalation phase has been completed, as in regime III, step 1468 (and step 1462). In a similar manner, other criteria may be used to trigger the turning on or off of the aerosol generator. For example, as described above, the start of aerosolization may be triggered by the sensing of a particular pressure or change in pressure in the ventilator circuit, and may end by following the turning off sequence of regimes III (steps 1468 and 1462) or IV (steps 1474, 1476, 1478 or 1480 and 1482, followed by step 1462, as described above.

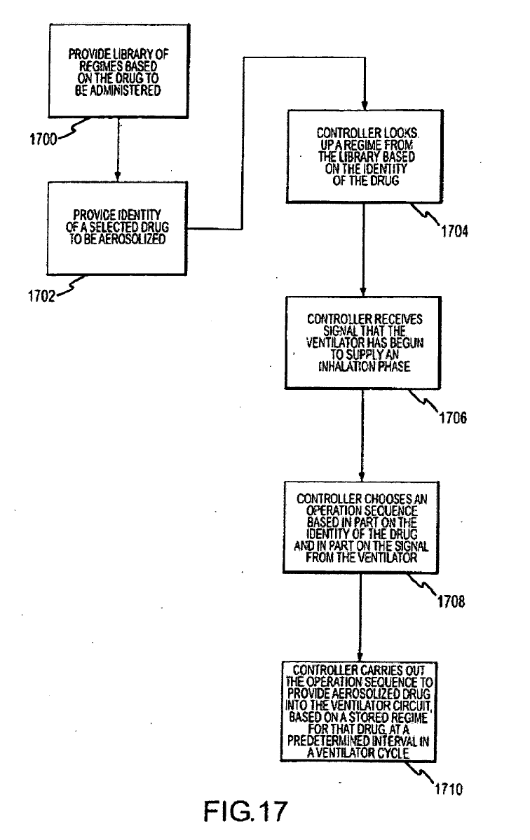

FIG. 17 is a schematic representation of an algorithm by which an operating sequence, for providing nebulized drug to a patient receiving air from a ventilator, may be chosen based on the combination of a plurality of independent sets of information, in this case, drug identity and a signal from the ventilator. In step 1700, a library of drug regimes is provided, the library based on various drugs that may be administered. In step 1702, the identity of a particular drug is provided to the system, and this may be provided, as described above, by a marker on a nebule containing the drug, the marker being read by the system. In step 1704, the controller looks up a regime from the library of stored regimes to select a regime based on the particular drug to be administered. In step 1706, the controller receives a signal from the ventilator. In step 1708, the controller then chooses an operation sequence based in part on the drug identity and drug regime and in part on the independent information provided by the signal from the ventilator. In step 1710, the controller carries out the operation sequence, which may be producing aerosol at a predetermined interval in the ventilation cycle based on the drug and the regime provided for the drug factored in with the inhalation cycle of the ventilator. These descriptions are illustrative, and accordingly, the order of the steps may be altered, and other variations, additions and modifications, as described above, may be made still in accordance with the present invention.

The phasic delivery methods outlined above may also be practiced with additional systems such as continuous positive airway pressure (“CPAP”) systems, such as the ones described in U.S. patent application Ser. No. 10/828,765, filed Apr. 20, 2004, U.S. patent application Ser. No. 10/883,115, filed Jun. 30, 2004, U.S. patent application Ser. No. 10/957,321, filed Sep. 9, 2004, where the entire continents of all the applications are herein incorporated by reference for all purposes.

One or more embodiments of the invention comprise systems and method for delivering relatively high concentrations of medicament without undue precipitation of the medicament in the delivery system. The on-vent adapter with the pattern of aerosol generation is designed to allow up to 90% of aerosol enter the artificial airway and allow 45-80% of dose to reach aerosol to the lungs. Hand Held adapter delivers inhaled mass of 75-90% with 40-55% of dose reaching the lungs as aerosol (based on AMIK phase II Scintigraphy.) Initially in vitro testing was showing precipitation of Vancomycin in powder form on the inspiratory filter. The innovation of the in vitro model with exhaled humidity, showed that precipitation would not be an issue in vivo.

### Experimental

### Aerosolized Antibiotic Lung Delivery Experiments

Delivery efficacy tests were conducted with an on-ventilator PDDS aerosolizing an aqueous solution of antibiotic (an aminoglycoside). The PDDS ventilator circuit configuration was similar to the one shown and described in FIG. 2 above. A 400 mg dose of the antibiotic was run through the PDDS. The PDDS was configured to deliver the aerosolized medicament by a phasic delivery regime similar to the one shown in FIG. 12C. The medicament dose was delivered over the course of about 50 to about 60 minutes.

Table 2 presents efficiency data for the delivery of aerosolized medicament to through systems according to embodiments of the invention. In the experimental setup, aerosolized droplets deposited on an inspiratory filter placed at a patient end interface are weighed and compared to the total weight of the dose of medicament that was aerosolized. The percentage of a dose deposited on the inspiratory filter represents the fraction of the total aerosolized dose that would be inhaled by a patient, and thus quantifies the efficiency of the system.

TABLE 2
Percent of Dose Deposited on Inspiratory Filter

| | Percent | | | |
|---|---|---|---|---|
| | Deposited on | | Standard | |
| Run No. | Filter | Mean | Deviation | % RSD |
| 1 | 69% | 71% | 0.04 | 6% |
| 2 | 75% | | | |
| 3 | 75% | | | |
| 4 | 77% | | | |
| 5 | 69% | | | |
| 6 | 66% | | | |
| 7 | 68% | | | |

Table 2 shows the efficiencies of 7 runs for a system according to an embodiment of the invention had a mean efficiency of 71%±6%. This efficiency level is well above conventional systems for the delivery of aerosolized medicaments, where the efficiency levels are typically 10% or less.

### Aerosolized Vancomycin Lung Delivery Experiments

Experimental measurements of lung deposition of aerosolized vancomycin were conducted that included modifications of conventional ventilator/nebulizer systems such as traps to collect fluid, positioning of a filter above the endotracheal tube (EU), and addition of heat and humidity to the circuit. The experimental analysis found an improved ability to differentiate aerosol from fluids delivered to the inspiratory filter, reduction in formation of precipitate, and reduction in the variance between conditions measured.

In some experiments improved conditions conducive to good aerosol delivery were found using the following delivery parameters:
Peak Insp Flow Rate: 40 Ipm
Tidal Volume 500 mL
Resp Rate: 15 bpm
Insp:Exp Ratio: 1:2

All tests were performed with a PB 7200AE ventilator with a descending (ramp) flow pattern and a bias flow of 6 Ipm. Heated humidification was provided by a heated humidifier, ConchaTherm III Plus (Tri-anim) with a 72-inch heated wire circuit (Tri-anim). Under dry conditions, the heated humidifier was bypassed, and a standard 72-inch non-heated wire circuit was used.

In all experiments the PDDS was placed at the proximal end of an 8.0 mm ID EU. The nebulizer placement was about 3 cm from the end of the EU. The length, angle and curvature of the EU were based on representative clinical conditions as described by Maclntyre in 2002. The inspiratory filter, was placed between the distal tip of the EU and a test lung (TTL, Michigan Instruments, MI) set to simulate a standard adult lung compliance (0.05 L/cm H2O) and resistance for the upper airway (5 cm H2O/L/sec) and the lower airway, (20 cm H2O/L/sec) unless otherwise stated.

All nebulizers used were pre-screened using normal saline to generate aerosol particles with a VMD of 4.0±0.2 um based on measurements by a light scattering laser diffraction instrument (Spraytec, Malven). The PDDS nebulizer generated aerosol during a defined fraction of inspiration (e.g., 75%) to deliver a nominal dose of 3.0 mL of Vancomycin containing 120 mg/ml in 0.25 normal saline. Ventilator readouts were recorded pre- and post-nebulization period. Three replicates were performed for each condition unless otherwise stated. For each test, the nebulizer was run until dryness.

### Experimental Test Set-Ups

Inspiratory filter—drug deposited distal to the airway
Expiratory filter—drug deposited in the expiratory limb of the ventilator circuit.
Nebulizer—drug remaining in nebulizer after nebulization is complete.
Nebulizer Tee—drug losses in nebulizer tee and EU adapter.
ETT—drug deposited in the artificial airway
Wye connector—drug deposited at the convergence of the inspiratory and expiratory limb of the ventilator circuit

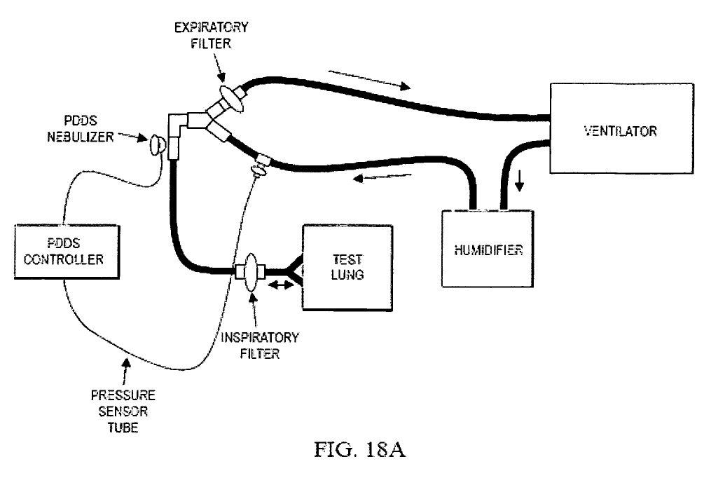

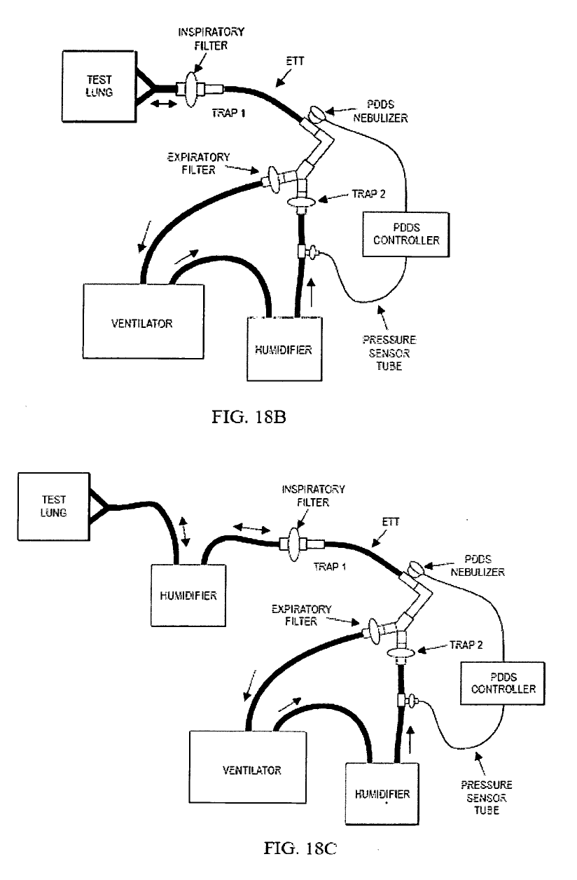

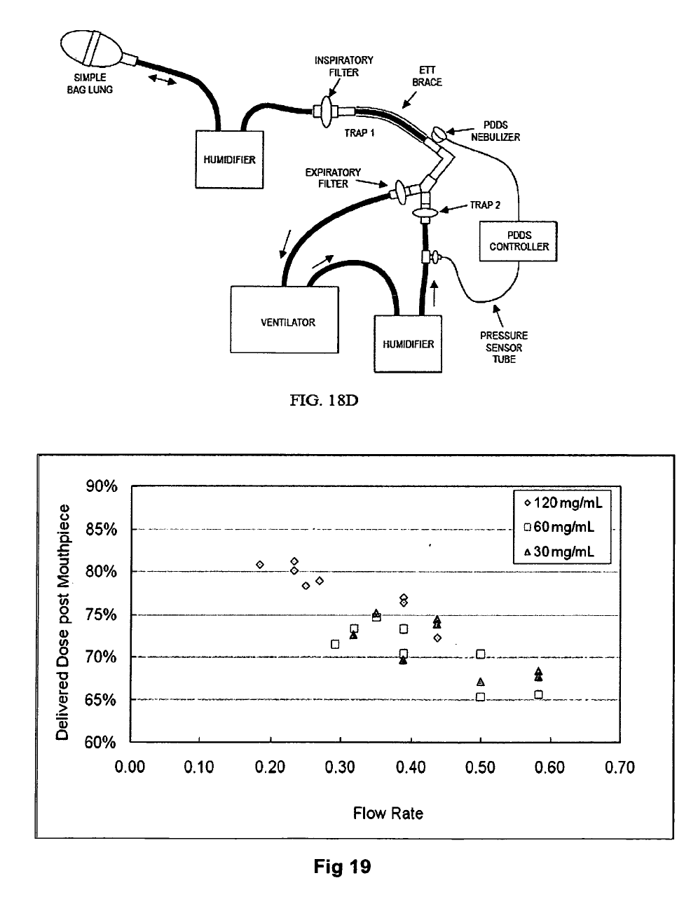

To separate aerosol dose fraction from condensate, two collection traps were added to the “classic” model (Setup 1, shown in FIG. 18A) with placement of the inspiratory filter above the EU and the nebulizer to create an 8-compartment model (Setup 2, FIG. 18B). The Figure shows the following numbered elements 1=inspiratory filter, 2=inspiratory trap, 3=EU, 4=T-fitting, 5=expiratory filter (condensate collector), 6=inspiratory filter (condensate collector), 7=nebulizer, and 8=Wye to inspiratory limb. Trap 1 was placed between the inspiratory filter and the distal tip of the EU to collect liquid leaving the EU. Trap 2 was placed in the inspiratory limb of the ventilator circuit positioned dependent to the nebulizer, to collect condensate from the EU and nebulizer tee.

Setup 3 (FIG. 18C) comprised an active heated humidifier (Conchatherm; RCI-Hudson) operated at 35° C., between the test lung and filter to simulate the temperature and absolute humidity conditions of patient exhalation through the ETT.

The final modification of the test setup, Setup 4, illustrated in FIG. 18D, focused on the improvement of drug loading capability, usability, and cost. A simple bag type test lung (Ambu) was used in place of the Michigan Instruments test lung (TTL), and disposable bacterial/viral filters (Vital Signs, Inc.) were used in place of the reusable filter (Pari) and its housing in order to simplify the chemical assay.

As the initial cause for modification of the in vitro setup was based on irregular results at high flow rates, setups 2, 3 and 4 were all initially tested at unfavorable conditions (PIFR of 80 Ipm, TV of 650 mL, RR of 12 BPM and I:E of 1:4) with ventilator circuit heat and humidity off.

Setup 4 was tested under favorable and unfavorable conditions with and without heated humidity in the ventilator circuit.

Vancomycin hydrochloride was eluted from filters and washed from compartments and determined by reverse phase HPLC with isocratic elution and UV detection at 280 nm. Mobile phase consisted of 92% TEA Buffer (0.2% TEA, pH 3.2), 7% Acetonitrile, 1% THF. The column (Agilent Extend-C18, Zorbax 80 Å, 4.6 mm×100 mm, 3.5 μm) was operated at ambient temperature. Flowrate was 1.75 mUmin with injection volume of 20 μL and run time of 8 min. Linear range of 5 mg/mL-0.1 mg/mL. Mass balance was determined. Results were expressed as mean±SD percent of initial dose of 360 mg.

All results were expressed as fraction of nominal dose delivered (mean±SD). Standard linear least squares fits of analysis of variance on each selected dependent variable (for example, inspiratory dose) using the independent parameters (PIFR and humidity) were performed with the JMP software package. Significant findings were identified as those with p-values of ≦0.05.

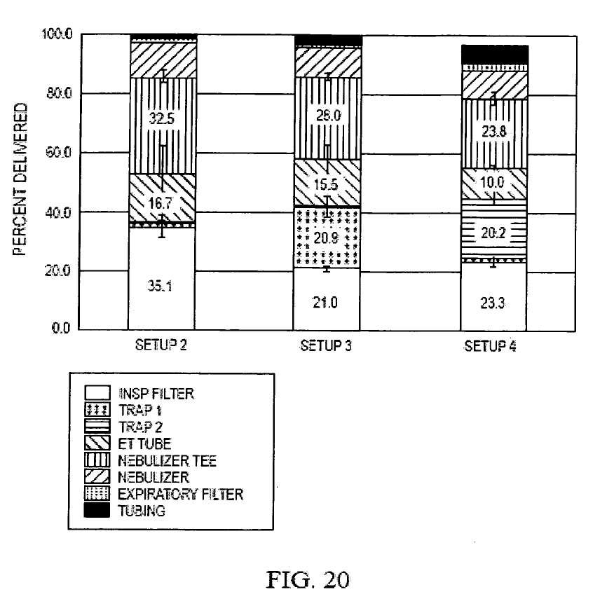

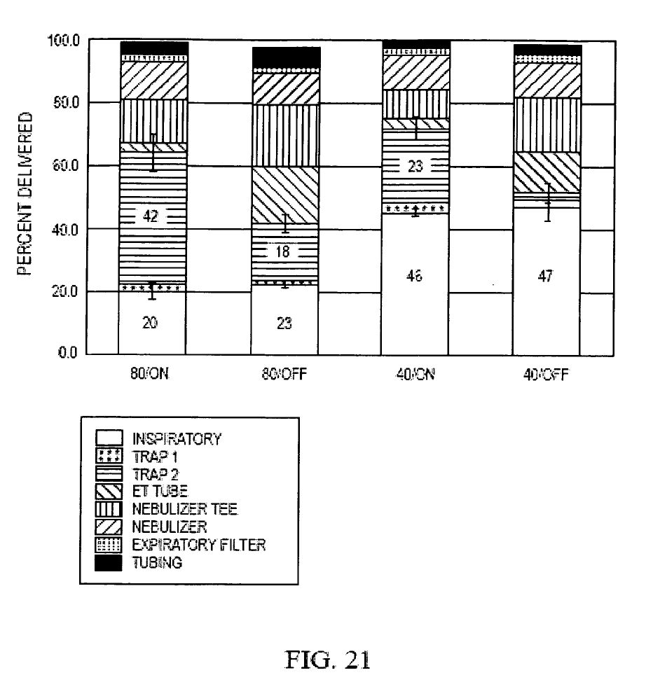

The distribution of drug in each compartment for setups 2, 3 and 4 is shown in FIG. 20. Flow rate and temperature conditions were considered unfavorable (i.e., 80 LPM and dry). The graph shows inspiratory aerosol dose with setup 3 and 4 are comparable, but both set-ups are significantly lower than setup 2 (without humidified exhalation.) In each of the three vertical bars shown, the percentage of drug collected from each compartment is plotted as follows: Starting from the bottom of the bar moving up, the plotted percentages represent the drug collected from: (1—bottom) the inspiratory filter, (2) Trap 1, (3) Trap 2, (4) Nebulizer Tee, (5) Nebulizer, (6) Expiratory Filter, and (7—Top) Tubing.

As FIG. 20 shows, the drug deposited on the inspiratory filter was significantly lower with humidified exhalation (Setup 3 and 4) than without (Setup 2; p<0.05). Comparable performance was found for both Setups 3 and 4. The R2 fit for the model was 0.90. Mass balance (summation of all test compartments) for each test run was >95%. Variability across inspiratory filter and ETT compartments was greater with Setup 2 than Setups 3 and 4.

The distributions of drug in compartments for Setup 4 under all four test conditions are shown in FIG. 21. In each of the four vertical bars shown, the percentage of drug collected from each compartment is plotted as follows: Starting from the bottom of the bar moving up, the plotted percentages represent the drug collected from: (1—bottom) the inspiratory filter, (2) Trap 1, (3) Trap 2, (4) ET tube (ETT), (5) Nebulizer tee, (6) Nebulizer (7) Expiratory Filter, and (8—Top) Tubing. The numeral under the bar is peak inspiratory flow rate (in Lpm) and “on” or “off” refers to humidification. The R2 for the inspiratory dose fit model was 0.97. This Figure shows that delivered dose tends to be dependent upon flow rate and independent of humidification.

The mass balance for each test run was greater than 95%. Results indicated that circuit humidity had negligible effect on inspiratory dose, contrary to claims in literature. There was no statistically significant difference in inspiratory dose deposition between wet and dry conditions for each flow rate. Significantly higher aerosol deposition was achieved at PIFR of 40 Lpm than for 80 Lpm (p<0.05). Inspiratory dose was about two fold higher at 40 Lpm than 80 Lpm under both dry and wet conditions. Furthermore, under dry conditions, minimal precipitate was observed to form on the inspiratory filter. Drug remaining in the EU and nebulizer tee were significantly greater under dry than wet conditions (p<0.05). In contrast, drug collected in trap 2 was significantly greater under wet than dry conditions (p<0.0001).

Placement of an active humidifier between the test lung and the inspiratory filter eliminated the difference in aerosol reaching the inspiratory filter between wet and dry conditions. In the conventional classic model, rainout of water is thought to occur as heated, humidified gas enters the test lung and cools. The gas exiting the test lung then exhibits lower water vapor content (absolute humidity) and temperature while retaining a high relative humidity. It is further though that the inhaled mass of aerosol in vitro is primarily a result of the mole fraction of water vapor in air (i.e. absolute humidity) rather than relative humidity. Thus large changes in absolute humidity from the ventilator circuit to and from the breathing simulator might impact aerosol as it passes through that transitional zone.

In one or more embodiments with the PDDS between the ventilator circuit and the ETT, no difference in aerosol deposition to the inspiratory filter was observed between wet and dry conditions when exhaled gas was heated and humidified.

In one or more embodiments placement of the inspiratory filter with a trap above the ETT enabled better discrimination of liquid aerosol versus liquid drug reaching the lungs.

Accordingly, in one or more embodiments of the present invention, active humidification may facilitate aerosol delivery with the aerosol generator placed at the ETT, and possibly under other conditions when the nebulizer is placed in the inspiratory limb.

It has thus been shown that in one or more embodiments of the present invention, placement of the nebulizer at the ETT advantageously can avoid dilution of aerosol with bias flow in the ventilator circuit. Increased fluid in the ETT during nebulization may result in the delivery of drug to the lungs exceeding the aerosol dose.

The test results suggest placement of the nebulizer close to the proximal end of the ETT and/or the use of active humidification can result in increased medicament (e.g., vancomycin) in trap 2. Thus, embodiments of the methods and systems of the present invention include the placement of the nebulizer close to the proximal end of the EU, for example within about 1 to about 5 cm of the proximal end (e.g., about 5 cm, about 4 cm, about 3 cm, about 2 cm or about 1 cm of the proximal end) and/or the use of active humidification can result in increased medicament (e.g., vancomycin) in trap 2. The reduction of drug in the ETT and nebulizer tee, suggests placement of the nubulizer near the proximal end of the ETT and active humidification results in more condensate and liquid forming in the nebulizer tee and ETT. Under clinical conditions, with the nebulizer and ETT superior to the patient, much of this drug containing liquid would likely be deposited in the lung, in addition to the inhaled aerosol.

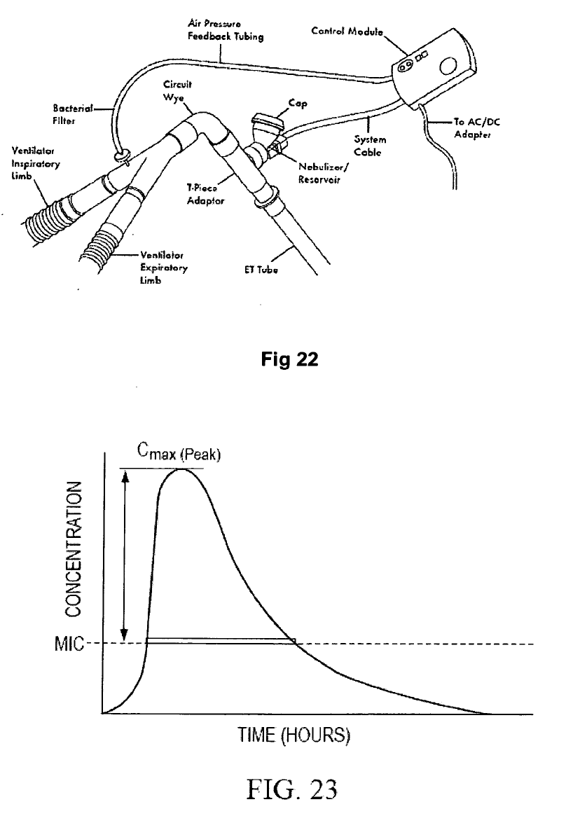

An exemplary configuration of placement of a nebulizer proximate to the proximal end of an ETT is shown in FIG. 22. The illustrated embodiment shows the nebulizer connected to the distal end of a circuit wye and proximal end of the ETT with a T-piece adaptor. As noted above, the nebulizer may be spaced within about 1 to about 5 cm of the proximal end (e.g., about 5 cm, about 4 cm, about 3 cm, about 2 cm or about 1 cm of the proximal end) of the ETT.

In one or more embodiments of methods and/or systems of the present invention, placement of the nebulizer at the airway allows aerosol administration with heat moisture exchangers (HMEs) in place. HMEs collect heat and moisture exhaled by the patient, transferring a substantial proportion of that heat and moisture to the next inhaled breath. Thus placement of the nebulizer proximal to the airway thus avoids the problem of nebulizer placement in the inspiratory limb of the ventilator circuit, requiring that the HME be removed to avoid filtering out the aerosol from inhaled gas.

In terms of the development of a drug delivery device platform, reducing the 40-50% variability between dry and wet conditions is a benefit. The more consistent the aerosol dosing between patients receiving mechanical ventilation across the range different conditions, the less the need to dictate limited or restrictive conditions to assure that an effective aerosol dose is delivered.

The use of active humidification to simulate highly saturated exhaled gas reduces the variability of inspiratory dose of aerosol between wet and dry conditions during mechanical ventilation with the PDDS nebulizer. The change in deposition across model components suggests that an intratracheally instilled dose of drug may be greater with wet as opposed to dry conditions and with higher inspiratory flow rates.

In one or more embodiments, there is thus no advantage in turning off heat and/or humidification during aerosol delivery via mechanical ventilation. In one or more embodiments, there is an advantage in providing humidification during aerosol delivery via mechanical ventilation.

FIG. 19 is a graph showing delivered dose of Vancomycin Hydrochloride at three different concentrations: 30, 60 and 120 mg/mL, as a function of flow rate. The test set-up was substantially as depicted in FIG. 18D (Setup 4) The vancomycin was delivered using a Nektar Therapeutics Handheld Aerosol Delivery System as depicted in FIGS. 2-3, and as described in U.S. Provisional Application No. 61/123,133. The nebulizer employed the Nektar tube core aerosol generator, substantially as described in PCT Patent Application Publication WO 2006/127181, and in FIGS. 9A-B and 10 herein. The nebulizer was filled with: 3.5 mL for each concentration. Flow rate was set on the breathing simulator. The nebulizer was allowed to operate until the fill volume of 3.5 mL was empty (typically about 15 minutes or less).

Samples were assayed using drug specific content method (HPLC) Conclusions: As flow rate increases, the Delivered dose post Mouthpiece decreases.

A dose-response to aerosolized vancomycin, administered in accordance with one or more embodiments of devices, apparatus and/or methods of the present invention is assessed. Also assessed is the ability to deliver a predefined target multiple of an MIC for Gram-positive bacteria causing pneumonia, including, Pseudomonas species, in tracheal aspirates (TA). Thus, one or more embodiments of devices, apparatus and/or methods of the present invention is used to deliver 2 or 3 or 4 or 5 or 10 or 15 or 20 or 25 or more times a reference MIC value for Gram-positive, pneumonia-causing bacteria.

For vancomycin susceptable S. aureas, the reference MIC is estimated at 2-4 μg/mL; 8-16 μg/mL for vancomycin intermediate S. aureas and 16-32 μg/mL or more for vancomycin resistant S. aureas.

In one or more embodiments, desired multiples, for example, are thus 4-8 μg/mL, 16-32 μg/mL, and 32-64 μg/mL, respectively for a 2×MIC concentration, and 8-16 μg/mL, 32-64 μg/mL, and 64-128 μg/mL, respectively for a 4×MIC concentration.

In one or more embodiments of the present invention peak serum concentrations should be below about: 40 μg/mL, or about 30 μg/mL or about 20 μg/mL or about 15 μg/mL or about 10 μg/mL or about 5 μg/mL and/or trough levels are below about 20 μg/mL, or about 15 μg/mL or about 10 μg/mL or about 8 μg/mL or about 5 μg/mL or about 3 μg/mL or about 2 μg/mL or about 1 μg/mL; and/or both. Aerosolized glycopeptide, such as vancomycin administered in accordance with one or more embodiments of devices, apparatus and/or methods of the present invention is expected to be well tolerated.

Conventional administration (intravenous and oral) of glycopeptides such as vancomycin, has been shown to require a multiple of MIC for a target organism of at least about 125, or 200, and as high as 400 or more for a therapeutic effect. In one or more embodiments of the present invention a therapeutic dose of aerosolized vancomycin is as low as 2 or 4 times the MIC for the same target organism. The present invention thus affords a dose reduction, compared to conventional administration, of at least about 20 times, such as at least about 31 times. In one or more embodiments of the present invention a therapeutic dose is about 50 or 62 or 100 or 200 times lower than a conventionally-administered dose.

Delivery of vancomycin in accordance with one or more embodiments of devices, apparatus and/or methods of the present invention thus is expected to provide safe serum concentrations, and to afford therapeutic lung concentrations with low serum levels. Moreover, use in this manner can reduce the need for systemically-administered (e.g. IV) antibiotics, especially in treating intubated patients with pulmonary and/or respiratory infections.

Having described several embodiments, it will be recognized by those of skill in the art that various modifications, alternative constructions, and equivalents may be used without departing from the spirit of the invention. Additionally, a number of well known processes and elements have not been described in order to avoid unnecessarily obscuring the present invention. Accordingly, the above description should not be taken as limiting the scope of the invention.

1. A method of administering a glycopeptide anti-infective to a patient, the method comprising the steps of:
converting the glycopeptide into an aerosol; and
delivering the aerosolized glycopeptide to the respiratory system of the patient, wherein a ratio of an amount of the glycopeptide delivered to the patient in a 24 hour period to a minimum inhibitory amount for the same period is about 2 or more.

2. The method of claim 1, wherein the ratio of the amount of the glycopeptide delivered to the patient in a 24 hour period to the minimum inhibitory amount for the same period is about 4 or more.

3. The method of claim 1, wherein the ratio of the amount of the glycopeptide delivered to the patient in a 24 hour period to the minimum inhibitory amount for the same period is about 10 or more.

4. The method of claim 1, wherein the glycopeptide is delivered intermittently to the patient.

5. The method of claim 4, wherein the intermittent delivery of the aerosolized glycopeptide comprises starting and stopping the delivery in each inhalation phase of a respiratory cycle of the patient.

6. The method of claim 4, wherein the intermittent delivery is via a ventilator circuit.

7. The method of claim 4, wherein the intermittent delivery is via a hand held aerosolization device.

8. The method of claim 4, wherein the intermittent delivery of the aerosolized comprises starting the delivery in each inhalation phase of a respiratory cycle of the patient, and stopping delivery in each exhalation phase of the respiratory cycle.

9. The method of claim 1, wherein the glycopeptide is converted into an aerosol using a vibrating mesh nebulizer.

10. The method of claim 1, wherein the glycopeptide is selected from vancomycin, dalbavancin, telavancin and combinations thereof.

11. The method of claim 10, wherein the glycopeptide comprises vamcomycin.

12. The method of claim 11, and further including an additional medicament selected from the group consisting of an antibiotic, an anti-oxidant, a bronchodialator, a corticosteroid, a leukotriene, a protease inhibitor, and a surfactant.

13. The method of claim 1 wherein the aerosolized vancomycin is delivered continuously

14. The method of claim 13, wherein the continuous delivery is via a ventilator circuit coupled to the respiratory system of the patient'

15. The method of claim 13, wherein the continuous delivery is into a hand held aerosolization device comprising an aerosolization chamber.

16-30. (canceled)

31. An aqueous composition for aerosolization, comprising:
an anti-infective comprising a glycopeptide, lipoglycopeptide or salt thereof present at a concentration from about 30 mg/mL to about 120 mg/mL, a pH of about 2.5 and 4.5, a viscosity of about 1.3 and 1.5 cSt, a surface tension of about 50 and 60 mN/m, a density of about 0.99 to 1.06 g/mL, and an osmolality of about 100 to 300 mMol/kg.

32. The aqueous composition of claim 31 wherein anti-infective comprises vancomycin.

33. (canceled)

34. A method of treating a pulmonary disease, the method comprising administering an aerosolized medicament wherein the medicament concentration in an epithelial lining fluid, or in tracheal aspirates, or both, exceeds a minimum inhibitory concentration for microorganisms usually responsible for Gram-positive pneumonia.

35. The method of claim 34 wherein the medicament comprises vancomycin.

36-37. (canceled)

38. The method of claim 37 wherein the vancomycin concentration is 200% of the minimum inhibitory concentration.

39-40. (canceled)
